(19) United States
(12) Patent Application Publication
(10) Pub. No.: US 2025/0090501 A1
(43) Pub. Date: Mar. 20, 2025

(54) METHODS OF TREATING GUT MOTILITY DISORDERS

(71) Applicant: THE REGENTS OF THE UNIVERSITY OF CALIFORNIA, Oakland, CA (US)

(72) Inventors: Faranak FATTAHI, Redwood City, CA (US); Homa MAJD, San Francisco, CA (US)

(73) Assignee: THE REGENTS OF THE UNIVERSITY OF CALIFORNIA, Oakland, CA (US)

(21) Appl. No.: 18/726,547

(22) PCT Filed: Jan. 3, 2023
(86) PCT No.: PCT/US2023/060053

§ 371 Date: Jul. 3, 2024

Related U.S. Application Data
(60) Provisional application No. 63/296,151, filed on Jan. 3, 2022.

(51) Int. Cl. A61K 31/4174; A61K 31/166; A61K 31/27; A61K 31/451; A61P 1/00

(52) U.S. Cl. CPC A61K 31/4174; A61K 31/166; A61K 31/27; A61K 31/451; A61P 1/00

(57) ABSTRACT

Compounds, pharmaceutical compositions, kits, and methods for treating gut motility disorders are herein. Examples of gut motility disorders to which the compounds, pharmaceutical compositions, kits, and methods are directed include achalasia, Hirschspmng's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence. This abstract is intended as a scanning tool for purposes of searching in the particular art and is not intended to be limiting of the present invention.

### CROSS REFERENCE TO RELATED APPLICATIONS

This application claims priority to U.S. Provisional Application Ser. No. 63/296,151, which was filed Jan. 3, 2022, in accordance with 35 USC 119 (e), and which is incorporated by reference in its entirety.

### STATEMENT REGARDING FEDERALLY SPONSORED RESEARCH

This invention was made with government support under grant numbers DP2NS116769 and R01CA240984 awarded by the National Institute of Health (NIH), and grant number R01DK121169 awarded by the National Institute of Diabetes and Digestive and Kidney Diseases. The government has certain rights in the invention.

### BACKGROUND

The enteric nervous system (ENS) is the largest and most complex division of the autonomic nervous system (De Giorgio, 2006). More than 500 million enteric neurons and roughly seven times as many enteric glia form interconnected enteric ganglia embedded in two distinct layers within the gut wall: the myenteric plexus residing between the longitudinal and circular muscles, and the submucosal plexus residing between the circular muscle and the mucosa (Grubišić and Gulbransen, 2017; Grundmann et al., 2019; Hamnett et al., 2021; Sasselli et al., 2012).

The ENS is not dependent on input from the central nervous system (CNS) to command gastrointestinal (GI) tract functions (Furness et al., 2014). This autonomy is exemplified by studies in which segments of the bowel removed from the body continue to generate complex motor patterns ex vivo. ENS autonomy is the result of extraordinarily diverse neuronal and glial cell types with distinct neurochemical signatures working together in harmony (Brehmer, 2021; Qu et al., 2008; Fung and Vanden Berghe, 2020). Thus, the ENS is equipped to control complex gut functions including motility, secretion, absorption, blood flow regulation, and barrier function support. Furthermore, the ENS communicates extrinsically with the CNS, enteroendocrine system, immune system, and the gut microbiome in order to maintain vitality and proper gut homeostasis (Furness et al., 2014; Long-Smith et al., 2020; Muller et al., 2014; Obata and Pachnis, 2016; Schneider et al., 2019; Yoo and Mazmanian, 2017).

The neurochemical and functional complexity of the ENS resembles the CNS (Gershon, 1999), yet much slower progress has been made in the field of ENS research. Despite being the largest and most complex division of the peripheral nervous system and playing a central role in the development and progression of enteric neuropathies and diseases of the gut-brain axis, ENS research has been disproportionately affected by multiple longstanding technical challenges. For example, enteric neurons are diluted throughout the GI tract, comprising less than 1% of gut tissue (Drokhlyansky et al., 2020). Therefore, scientists must rely on samples collected during GI resection surgeries, rather than more routine GI biopsies, to access ENS tissue. Furthermore, it is difficult to isolate the ENS without significant sampling bias related to harsh tissue dissociation techniques that damage fragile neurites, and reliable surface markers suitable for FACS purification of enteric neurons and glia are lacking.

The complex developmental processes and the elaborate cellular architecture of the ENS, as well as its remarkable communication with the rest of the body provide a wide array of possibilities for abnormalities to arise. Comprising some of the most challenging clinical disorders, enteric neuropathies, also known as disorders of gut brain interaction (DGBI), result from loss, degeneration, or functional impairment of the ENS cell types (De Giorgio et al., 2016; Niesler et al., 2021). This incomplete understanding of ENS development and function is accountable for the long-term morbidity and mortality of GI disorders and limited availability of therapeutic interventions. Specifically, there has been an extraordinary interest in gaining a better understanding of enteric nitrergic neurons (NO neurons) that release nitric oxide (NO) to relax the smooth muscle tissue and promote GI motility. This is due to the selective dysfunction, and degeneration of NO neurons in different forms of DGBI including, esophageal achalasia, infantile hypertrophic pyloric stenosis and gastroparesis (Bódi et al., 2019; Rivera et al., 2011). Accordingly, there remains a need for compounds and compositions capable of modulating NO neuron activity and methods of making and using same.

### SUMMARY

In accordance with the purpose(s) of the disclosure, as embodied and broadly described herein, the disclosure, in some embodiments, relates to compounds and compositions useful in the treatment of gut motility disorders such as, for example, achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

Thus, provided herein are methods for treating a gut motility disorder in a subject in need thereof, the method comprising administering to the subject an effective amount of compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof.

In some embodiments, the compound is selected from aripiprazole, dexmedetomidine, matrine, and MPEP, or a pharmaceutically acceptable salt thereof. In some embodiments, the compound is dexmedetomidine. In some embodiments, the gut motility disorder is selected from achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence. In some embodiments, the subject is a mammal. In some embodiments, the mammal is a human. In some embodiments, the subject has been diagnosed with a need for treatment of the gut motility disorder prior to the administering step. In some embodiments, the method further comprises identifying the subject in need of treatment of a gut motility disorder. In some embodiments, the effective amount is a therapeutically effective amount. In some embodiments, the effective amount is a prophylactically effective amount.

Also provided are methods of modulating NO neuron activity in a subject having a gut motility disorder, the method comprising administering to the subject an effective amount of a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof.

In some embodiments, the compound is selected from aripiprazole, dexmedetomidine, matrine, and MPEP, or a pharmaceutically acceptable salt thereof. In some embodiments, the compound is dexmedetomidine. In some embodiments, modulating NO neuron activity induces colonic activity.

Also provided are kits comprising a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, and one or more selected from: (a) an agent known for treating a gut motility disorder; (b) instructions for treating a gut motility disorder; and (c) instructions for administering the compound in connection with treating a gut motility disorder.

In some embodiments, the agent is selected from a parasympathomimetic, a prokinetic agent, an opiod antagonist, an antidarrheal, and an antibiotic. In some embodiments, the agent is selected from neostigmine, bethanechol, metoclopramide, cisapride, and loperamide. In some embodiments, the compound and the agent are co-packaged. In some embodiments, the compound and the agent are co-formulated.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE FIGURES

The accompanying figures, which are incorporated in and constitute a part of this specification, illustrate several aspects and together with the description serve to explain the principles of the disclosure.

FIG. 1A-H show representative data illustrating the identification of enteric NO neuron modulators by functional high-throughout screenings. Specifically, FIG. 1A shows flow cytometry quantification of stage 2 enteric ganglioid cFOS expression in response to epinephrine. Mean and SEM error bars are shown, ****: p-value <0.0001. FIG. 1B-C show multi-electrode array (MEA) analysis (FIG. 1B) and quantification of changes in neuronal firing (FIG. 1C) in response to epinephrine in stage 1 enteric ganglioids. FIG. 1D shows the identification of candidate neuromodulators that induce cFOS expression in stage 2 enteric ganglioid NO neurons by HTS. FIG. 1E shows the identification of candidate neuromodulators that induce NO release in stage 1 2D ENS culture NO neurons by HTS. FIG. 1F shows the predicted targets and FDA approval status of hits identified in cFOS induction and NO release screens. FIG. 1G shows a snRNA-seq analysis violin plot of module scoring for all predicted NO neuron activity promoting receptor genes in stage 1 enteric ganglioid NO subtypes versus other neurons. FIG. 1H shows a snRNA-seq analysis dot plot of the average module scores for individual categories of NO neuron activity promoting receptor gene categories.

FIG. 2A-N show representative data illustrating that NO neuron specific functional screening identifies modulators of colonic motility. Specifically, FIG. 2A shows a schematic representation of a high-throughput flow cytometry-based screening to identify compounds that induce cFOS expression in hESC-derived stage 2 enteric ganglioid NO neurons. FIG. 2B shows the target classes of the hits identified in enteric NO neuron cFOS induction screening (FIG. 1D, red dots). FIG. 1C shows a schematic representation of a high-throughput calorimetry-based screening to identify compounds that induce NO release in hESC-derived stage 1 2D ENS cultures. FIG. 2D shows the target classes of the hits identified in NO release screening (FIG. 1E, red dots). Protein classes that are in common with (FIG. 2B) are indicated with asterisks. FIG. 1E shows feature plots showing the predicted responsiveness of subclustered stage 1 ganglioid enteric NO neurons to neurotransmitters by module scoring of neurotransmitter receptor gene families. FIG. 2F shows a dot plot of the expression of genes belonging to the target classes shown in FIG. 2B and FIG. 2D in hESC-derived stage 1 and primary human enteric nitrergic neuron subtypes versus all other neurons. FIG. 2G shows the combined protein target analysis for selected screening hits showing shared protein classes. Color code matches the target classes in FIG. 2B and FIG. 2D. FIG. 2H shows a schematic representation of testing the effect of selected candidate hits (listed in (FIG. 2G) on mouse colonic motility ex vivo. FIG. 2I shows a representative spatiotemporal map of mouse colon contractions along the proximal-distal axis over a 10-min period. FIG. 2J shows the quantification of colonic migrating motor complexes (CMMC) intervals at 75th percentile of CMMC cumulative percentage (FIG. 3A) for selected hit compounds. FIG. 2K shows the experimental design for measuring the effect of selected candidate hits on mouse colonic motility ex vivo. Representative spatiotemporal maps of a mouse colon contraction along the proximal-distal axis over a 26-min period. Three representative longitudinal contractile events (LCEs) are shown per condition (arrows). FIG. 2L shows diagrams of CMMC cumulative percentile and quantification of CMMC interval (time difference between two consecutive contractions) at 75th percentile for dexmedetomidine. Mean and SEM error bars for 5 pairs of untreated and drug-treated mouse colons are shown. FIG. 2M shows the total number of colonic longitudinal contractile events (LCEs) within each 6-min treatment condition for 5 dexmedetomidine-treated mouse colons measured from spatiotemporal maps. *: p-value<0.05. FIG. 2N shows the mean of LCE duration calculated for three LCEs within each 6-min treatment (1 in the beginning, 1 in the middle, and 1 in the end of each spatiotemporal map, see (FIG. 2K)). Data are shown for 5 dexmedetomidine-treated mouse colons. SEM error bars are shown.

FIG. 3A-C show representative data illustrating the testing of selected HTS hits in ex vivo mouse colonic motility assays. Specifically, FIG. 3A-B show diagrams of CMMC (FIG. 3A) and slow wave (FIG. 3B) cumulative percentiles. FIG. 3C shows the quantification of latency between slow waves at 75th percentile for selected HTS hits; from left to right: aripiprazole, dexmedetomidine, latrepirdine, matrine, MPEP and naproxen. One-directional SEM error bars are shown in FIG. 3A-B.

FIG. 4A-F show representative data illustrating the effect of candidate drugs on ex vivo mouse colonic migrating motor complexes (CMMCs). Specifically, FIG. 4A-F show diagrams of CMMC cumulative percentile and quantifications of CMMC intervals at 75th percentile for aripiprazole (FIG. 4A-B), matrine (FIG. 4C-D), and MPEP (FIG. 4E-F). Compounds were used at 1 μM and mean and one directional SEM error bars are shown in FIG. 4A, FIG. 4C, and FIG. 4E.

FIG. 5A-B show representative data illustrating the effect of candidate drugs on ex vivo mouse colonic CMMC and slow wave intervals at 75th percentile. Specifically, FIG. 5A-B show quantifications of CMMC (FIG. 5A) and slow wave (FIG. 5B) intervals (time difference between two consecutive contractions) at 75th percentile for dexmedetomidine, aripiprazole, matrine, and MPEP. In each panel, top rows (blue) show control colons and bottom rows (red) show treatment colons within each pair. Compounds were used at 1 μM.

FIG. 6A-H show representative data illustrating the effect of candidate drugs on ex vivo mouse colonic slow waves. Specifically, FIG. A-H show diagrams of slow wave cumulative percentile and quantifications of latency between slow waves at 75th percentile for aripiprazole (FIG. 6A-B), dexmedetomidine (FIG. 6C-D), matrine (FIG. 6E-F), and MPEP (FIG. 6G-H). Compounds were used at 1 μM and mean and one-directional SEM error bars are shown in FIG. 6A, FIG. 6C, FIG. 6E, and FIG. 6G.

FIG. 7A-P show representative data illustrating that PDGFR inhibition promotes enteric NO neuron induction. Specifically, FIG. 7A shows a schematic representation of a high-throughput pharmacological screening to identify compounds that enrich NO neurons in hESC-derived 2D ENS cultures. FIG. 7B shows a combined protein target analysis for the HTS top 12 hits showing shared protein classes between structurally similar hits. FIG. 7C shows the effect of PP121 treatment window on NOS1::GFP induction efficiency. FIG. 7D shows immunofluorescence staining of NOS1 and neuronal TUBB3 in stage 1 enteric gangliods treated with or without PP121 between days 15 and 20. FIG. 7E shows a split UMAP of cell types present in stage 1 control (top) and PP121 treated (bottom) enteric gangliod cultures. FIG. 7F shows a dot plot of the average module scores of control only enteric gangliod subtype transcriptional signatures in PP121 treated gangliodl subtypes. FIG. 7G shows a splut UMPA of neuronal subtypes present in stage 1 control (top) and PP121 treated (bottom) enteric gangliod cultures. FIG. 7H shows a dot plot of the average modules scores of control only neuronal subtype transcriptional signatures in PP121 treated gangliod neuronal subtypes. FIG. 7I shows a distribution of NO neuron subtypes in control versus PP121 treated stage 1 enteric gangliod cultures. FIG. 7J shows a splut UMAP of subclustered NO subtypes present in stage 1 control (top) and PP121 treated (bottom) enteric gangliod cultures. FIG. 7K shows a dot plot of the average module scores of control only NO neuron subtype transcriptional signatures in PP121 treated gangliod NO neuron subtypes. FIG. 7L shows a feature plot showing the expression of ERBBs, PDGFRs, and VEGFRs in D15 subclustered enteric crestospheres. FIG. 7M shows a schematic of receptor tyrosine kinase (RTK) natural agonists and selected pharmacological antagonist including NO neuron enriching top hit PP121. FIG. 7N shows the effect of RTK ligand treatment on stage 1 enteric gangliod NO neuron induction. FIG. 7O and FIG. 7P show the effect of knocking out PDGFRA (FIG. 7O) and PDGFRB (FIG. 7P) in D15 enteric crestospheres on stage 1 enteric gangliod NO neuron enrichment as measured by flow cytometry

FIG. 8A-D show representative data illustrating that small molecue high-throughput screening identifies compounds that enrich NO neurons in hESC-derived ENS clutures. Specifically, FIG. 8A illustrates the identification of candidate compounds that enrich NO neurons in stage 1 2D ENS cultures. FIG. 8B shows HTS hits with a >8.0 fold increase in % NOS1+ cells. FIG. 8C and FIG. 8D show that PP121 induction of NOS1 expression is dose and time dependent. Representative flow diagrams showing NOS1 expression in stage 1 enteric neurons when cultures were treated with different PP121 concentrations at D15-D20 (FIG. 8C) or with 2 μM PP121 over different time-windows (FIG. 8D).

FIG. 9A-F show representative data illustrating that PP121 treatment enriches NO neurons without affecting their overall cellular diversity. Specifically, FIG. 9A shows the distribution of cell types in control versus PP121 treated stage 1 enteric ganglioid cultures. FIG. 9B shows the correlation of average gene expression in matched control versus PP121 treated enteric ganglioid cell types. FIG. 9C shows the distribution of neuronal subtypes in control versus PP121 treated stage 1 enteric ganglioid cultures. FIG. 9D shows the correlation of average gene expression in matched control versus PP121 treated enteric ganglioid neuronal subtypes. FIG. 9E shows the distribution of NO neuron subtypes in control versus PP121 treated stage 1 enteric ganglioid cultures. FIG. 9F shows the correlation of average gene expression in matched control versus PP121 treated enteric ganglioid NO neuron subtypes.

FIG. 10A-E show representative data illustrating that a human surface marker antibody screening identifies NO neuron specific surface markers. Specifically, FIG. 10A shows a schematic representation of using enriched enteric NO neuron cultures to identify their specific surface markers by a flow cytometry-based high-throughput antibody screening. FIG. 10B shows that surface markers that were expressed in at least 50% of NOS1+ cells, i.e., showing >50% sensitivity (top) were filtered based on their specificity for NOS1+ cells (at least 70% of all stained cells being NOS1+, i.e., >70% specificity, middle). FIG. 10C shows the surface marker screening top hits with the highest specificity as well as sensitivity for identifying enteric NO neurons. FIG. 10D shows a Violin plot stack showing the expression (left) and dot plot showing the average scaled expression (right) of surface marker screen hits in control and PP121 treated NO neuron subtypes versus other neurons. FIG. 10E shows a Violin plot stack showing the expression of surface marker screen hits in adult human NO neuron subtypes versus other neurons. FIG. 10F shows an immunofluorescence analysis for expression of neuronal marker TUBB3, NOS1, and CD47 in adult human primary colon tissue. A myenteric plexus ganglion is shown. Arrows show colocalization of CD47 and NOS1. FIG. 10G shows surface markers that stained at least 70% of total cells as potential pan ENS cell markers. FIG. 10H shows CD24 expression in stage 1 enteric ganglioid clusters in PP121 treated, and untreated samples. FIG. 10I shows a representative immunofluorescence analysis of CD24 and neuronal marker TUBB3 expression in a human primary colonic section, showing a myenteric plexus ganglion.

FIG. 11A-C show representative data illustrating the extensive engraftment of hESC-derived gangliods in adult mouse colon. Specifically FIG. 11A shows a schematic showing transplantation of hESC-derived stage 1 enteric ganglioids into mouse proximal colon. FIG. 11B shows the engraftment of hESC-derived stage 1 enteric ganglioid cells into the entire length of mouse colon as shown by the expression of human cytoplasmic marker SC121 in red. FIG. 11C shows an immunohistochemical analysis of human cytoplasmic protein SC121, and NO neuron marker NOS1 in Nos1−/− mouse colon 8 weeks post transplantation.

FIG. 12 shows representative data illustrating hESC-derived enteric gangliods engraft in adult mouse colon. Specifically, immunohistochemical analysis of neuronal TUBB3, human cytoplasmic protein SC121, and NO neuron marker NOS1 in Nos1−/− mouse colon (untreated with Cyclosporin A and non-operated, top) and 8 weeks post transplantation (bottom) is shown. Purple arrows point at NOS1+ cells.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The present invention can be understood more readily by reference to the following detailed description of the disclosure and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc.

The term “or” as used herein shall only be interpreted as indicating exclusive alternatives (i.e., “one or the other but not both”) when preceded by terms of exclusivity, “either,” “one of,” “only one of,” or “exactly one of.”

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated with NO neuron activity such as, for example, a gut motility disorder, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In some embodiments, an IC₅₀ can refer to the concentration of a substance that is required for 50% inhibition in vivo, as further defined elsewhere herein.

As used herein, “EC₅₀” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve 50% of the maximum response in vivo, as further defined elsewhere herein.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain some embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl) alkyl or (cycloalkyl) alkenyl.

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “heteroalkyl,” as used herein, refers to an alkyl group containing at least one heteroatom. Suitable heteroatoms include, but are not limited to, O, N, Si, P, and S, wherein the nitrogen, phosphorous and sulfur atoms are optionally oxidized, and the nitrogen heteroatom is optionally quaternized. Heteroalkyls can be substituted as defined above for alkyl groups.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partically saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro [4.5]decanyl, spiro [4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cycloheptyl, cyclooctyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein. In various aspects, the cycloalkyl group and heterocycloalkyl group can be monocyclic, bicyclic (e.g., bridged such as, for example, bicyclo[4.3.1]decanyl or spiro such as, for example, spiro[3.6]decanyl, spiro[4.5]decanyl, spiro[4.4]nonyl), polycyclic (e.g., tricyclic), or a fused hydrocarbon ring system that is saturated or partially unsaturated (e.g., decahydronaphthalenyl, dihydroindenyl, decahydroazulenyl, octahydroazulenyl, tetrahydronaphthalenyl).

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” The heterocycle can be monocyclic, bicyclic (e.g., spiro or bridged), polycyclic, or a fused system that is saturated or partially saturated. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2, or 3 ring heteroatoms or wherein a pyridine ring is fused to a 5- or a 6-membered ring containing 1, 2, or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals comprising at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “aromatic group” as used herein refers to a ring structure having cyclic clouds of delocalized π electrons above and below the plane of the molecule, where the x clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein, refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl) amino group, (tert-butyl) amino group, pentylamino group, isopentylamino group, (tert-pentyl) amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di (sec-butyl) amino group, di (tert-butyl) amino group, dipentylamino group, diisopentylamino group, di (tert-pentyl) amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain some embodiments, unless expressly indicated to the contrary, individual substituents can be further optionally substituted (i.e., further substituted or unsubstituted).

In some embodiments, a structure of a compound can be represented by a formula:

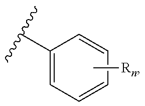

which is understood to be equivalent to a formula:

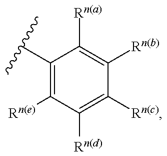

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

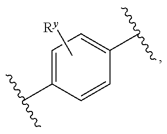

wherein R^{y} represents, for example, 0-2 independent substituents selected from A¹, A², and A³, which is understood to be equivalent to the groups of formulae:
   wherein R^{y} represents 0 independent substituents

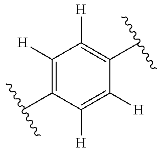

wherein R^{y} represents 1 independent substituent

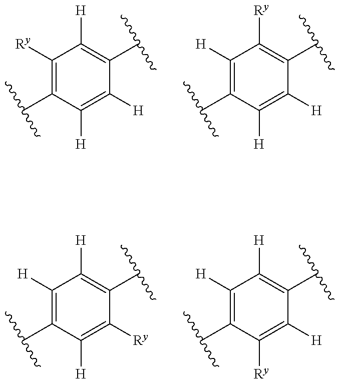

wherein R^{y} represents 2 independent substituents

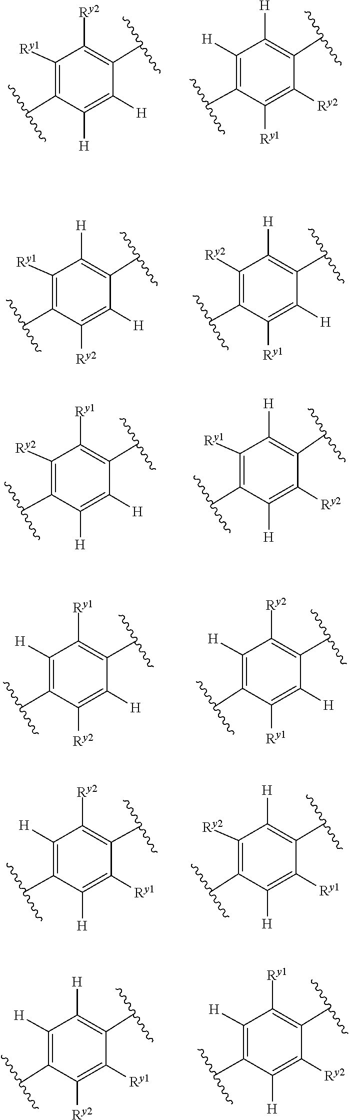

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

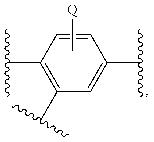

wherein, for example, Q comprises three substituents independently selected from hydrogen and A, which is understood to be equivalent to a formula:

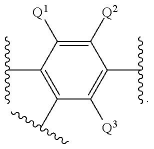

Again, by “independent substituents,” it is meant that each Q substituent is independently defined as hydrogen or A, which is understood to be equivalent to the groups of formulae:
wherein Q comprises three substituents independently selected from H and A

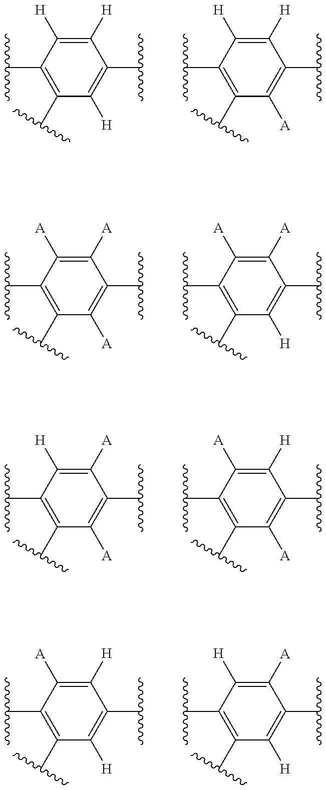

In some embodiment, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least about 60%, 70%, 80%, 90%, 99%, or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Disease, disorder, and condition are used interchangeably herein.

As used herein, the terms “treatment,” “treat,” and “treating” refer to reversing, alleviating, delaying the onset of, reducing, ameliorating, preventing, or inhibiting the progress of a disease or disorder, or one or more symptoms thereof, as described herein. “Treating” may refer to the administration of the compounds, compositions, or pharamaceutical compositions described herein. “Treating” includes the concepts of “alleviating”, which refers to lessening the frequency of occurrence or recurrence, or the severity, of any symptoms or other ill effects related to a gut motility disorders. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “therapeutic effect” as used herein is meant to refer to some extent of relief of one or more of the symptoms of a disorder (e.g., a gut motility disorder) or its associated pathology. The term “effective amount” or “therapeutically effective amount” refers to an amount that is sufficient to achieve the desired result (e.g., that will elicit a biological or medical response of a subject e.g., a dosage of between 0.01-100 mg/kg body weight/day) or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition.

As used herein, the term “sample” refers generally to a limited quantity of something which is intended to be similar to and represent a larger amount of that something. In the present disclosure, a sample is a collection, swab, brushing, scraping, biopsy, removed tissue, or surgical resection that is to be testing for the absence, presence or grading of a gut motility disorder. In some embodiments, samples are taken from a patient or subject that is believed to have a gut motility disorder.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “associated” or “associated with” in the context of a substance or substance activity or function associated with a disease (e.g., a protein associated disease, a symptom associated with a gut motility disorder, a symptom associated with NO neuron activity) means that the disease (e.g., the gut motility disorder) is caused by (in whole or in part), or a symptom of the disease is caused by (in whole or in part) the substance or substance activity or function. For example, a symptom of a gut motility disease or condition may be a symptom that results (entirely or partially) from modulation of NO neuron activity (e.g., induction of colonic motility). As used herein, what is described as being associated with a disease, if a causative agent, could be a target for treatment of the disease. For example, a gut motility disorder, may be treated with an agent (e.g., compound as described herein) effective for modulating NO neuron activity (e.g., effective for inducing colonic motility).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g., PINK1). In some embodiments contacting includes allowing a compound described herein to interact with a protein or enzyme that is involved in a signaling pathway.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the remainder of a molecule or chemical formula.

As defined herein, the term “activation,” “activate,” “activating,” and the like in reference to a protein-activator (e.g., agonist) interaction means positively affecting (e.g., increasing) the activity or function of the protein relative to the activity or function of the protein in the absence of the activator. In some embodiments, activation refers to an increase in the activity of a signal transduction pathway or signaling pathway. Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein that may modulate the level of another protein or increase cell survival.

The term “modulator” refers to a composition that increases or decreases the level of a target molecule or the function of a target molecule. In some embodiments, the modulator is a modulator of NO neuron activity. In some embodiments, the modulator is a modulator of NO neuron activity and is a compound that reduces the severity of one or more symptoms of a disease associated with NO neuron activity. In some embodiments, a modulator is a compound that reduces the severity of one or more symptoms of a gut motility disorder that is not caused or characterized by NO neuron activity but may benefit from modulation of NO neuron activity (e.g., induction of colonic motility).

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with a compound, pharmaceutical composition, or method provided herein. In some embodiments, the disease is a disease related to (e.g., characterized by) modulation of NO neuron activity. In some embodiments, the disease is a gut motility disorder.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of a gut motility disorder), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a gut motility disorder as further described herein, or with adjunctive agents that may not be effective alone, but may contribute to the efficacy of the active agent.

In some embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In some embodiments, the active and/or adjunctive agents may be linked or conjugated to one another. In some embodiments, the compounds described herein may be combined with treatments for neurodegeneration such as surgery. In some embodiments, the compounds described herein may be combined with treatments for cardiomyopathy such as surgery.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In some embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In an embodiment, a triphosphate derivative is a gamma-thio triphosphate. In an embodiment, a derivative is a phosphoramidate. In some embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vernachio et al., ACC, 2011, 55, 1843; Zhou et al, AAC, 2011, 44, 76; Reddy et al., BMCL, 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov., 2008, 7, 255, all of which are incorporated herein by reference in their entirety for all purposes.

As used herein, the term “animal” includes, but is not limited to, humans and non-human vertebrates such as wild, domestic, and farm animals.

As used herein, the term “antagonize” or “antagonizing” means reducing or completely eliminating an effect.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “individual,” “subject,” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the disclosure can optionally exclude one or more embodiment for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

### B. Methods of Treating a Gut Motility Disorder

In some embodiments, compounds and compositions described herein are useful in treating a gut motility disorder. Thus, provided herein are methods of treating a gut motility disorder, comprising administering to a subject in need thereof, a therapeutically effective amount of a compound as described herein (e.g., modulators of COX, dopamine receptors, sodium channels, serotonin receptors, acetylcholine receptors, GABA receptors, FAAH, adrenergic receptors, histamine receptors, vasopressin receptors, NMDAR, beta amyloid, gamma-secretase, IxB/IKK, glutamate receptors, opioid receptors, TRPV, aldose reductase, calcium channels, glucocorticoid receptors, and/or HMG-COA reductase) or a pharmaceutically acceptable salt thereof, or a composition comprising a disclosed compound or pharmaceutically acceptable salt thereof. Disorders treatable by the present compounds and compositions include, e.g., achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

In some embodiments, the disclosure relates to any of the above disclosed methods disclosed herein, wherein the administerating step comprises administering a pharmaceutical composition comprising: (i) a pharmaceutically effective amount of any of the disclosed compounds; and (ii) a pharmaceutically acceptable carrier.

Thus, in various embodiments, disclosed are methods for treating a gut motility disorder in a subject in need thereof, the method comprising administering to the subject an effective amount of a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof. In a further embodiment, the compound is selected from aripiprazole, dexmedetomidine, matrine, and MPEP, or a pharmaceutically acceptable salt thereof. In still further embodiments, the compound is dexmedetomidine.

In further embodiments, the compound is FDA approved.

In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In various embodiments, the method further comprises administering an effective amount of an agent associated with the treatment of a gut motility disorder.

Thus, in various embodiments, the method further comprises administering an agent known for the treatment of a gut motility disorder. Examples of agents known for the treatment of gut motility disorders include, but are not limited to, parasympathomimetics, prokinetic agents (also called promotility agents), opiod antagonists, antidarrheals, and antibiotics. In further embodiments, the agent is selected from neostigmine, bethanechol, metoclopramide, cisapride, and loperamide.

In some embodiments, the compound and the agent are administered simultaneously. In some embodiments, the compound and the agent are administered sequentially.

In some embodiments, the compound and the agent are co-packaged. In some embodiments, the compound and the agent are co-formulated.

### C. Methods of Modulating No Neuron Activity in a Subject

In some embodiments, disclosed are methods of modulating NO neuron activity in a subject, the method comprising the step of administering to the subject a therapeutically effective amount of at least one disclosed compound (e.g., modulators of COX, dopamine receptors, sodium channels, serotonin receptors, acetylcholine receptors, GABA receptors, FAAH, adrenergic receptors, histamine receptors, vasopressin receptors, NMDAR, beta amyloid, gamma-secretase, IxB/IKK, glutamate receptors, opioid receptors, TRPV, aldose reductase, calcium channels, glucocorticoid receptors, and/or HMG-COA reductase), or a pharmaceutically acceptable salt thereof.

Thus, in various embodiments, disclosed are methods for modulating NO neuron activity in a subject having a gut motility disorder, the method comprising administering to the subject an effective amount of a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof. In a further embodiment, the compound is selected from aripiprazole, dexmedetomidine, matrine, and MPEP, or a pharmaceutically acceptable salt thereof. In a still further embodiment, the compound is dexmedetomidine.

In further embodiments, modulating NO neuron activity induces colonic activity.

As used herein, “modulation” can refer to either inhibition or enhancement of a specific activity. For example, the modulation of NO neuron activity can refer to the inhibition and/or activation of NO neuron dependent activities, such as an increase in colonic motility. In some embodiments, the compounds described herein increase colonic motility by a factor from about 1% to about 50%. The activity of NO neurons can be measured by any method including but not limited to the methods described herein.

In further embodiments, modulating is inducing colonic motility.

In further embodiments, the subject is a mammal. In still further embodiments, the subject is a human.

In further embodiments, the subject has been diagnosed with a need for treatment of a gut motility disorder prior to the administering step. In still further embodiments, the method further comprises the step of identifying a subject at risk of developing a gut motility disorder prior to the administering step.

In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In various embodiments, the method further comprises administering an effective amount of an agent associated with the treatment of a gut motility disorder.

Thus, in various embodiments, the method further comprises administering an agent known for the treatment of a gut motility disorder. Examples of agents known for the treatment of gut motility disorders include, but are not limited to, parasympathomimetics, prokinetic agents, opiod antagonists, antidarrheals, and antibiotics. In further embodiments, the agent is selected from neostigmine, bethanechol, metoclopramide, cisapride, and loperamide.

In some embodiments, the compound and the agent are administered simultaneously. In some embodiments, the compound and the agent are administered sequentially.

In some embodiments, the compound and the agent are co-packaged. In some embodiments, the compound and the agent are co-formulated.

### D. Pharmaceutical Compositions

Also provided herein are pharmaceutical compositions comprising a compound as disclosed herein, or pharmaceutically acceptable salt thereof; and a pharmaceutically acceptable carrier. Thus, in various embodiments, disclosed are pharmaceutical compositions comprising a therapeutically effective amount at least one disclosed compound (e.g., modulators of COX, dopamine receptors, sodium channels, serotonin receptors, acetylcholine receptors, GABA receptors, FAAH, adrenergic receptors, histamine receptors, vasopressin receptors, NMDAR, beta amyloid, gamma-secretase, IxB/IKK, glutamate receptors, opioid receptors, TRPV, aldose reductase, calcium channels, glucocorticoid receptors, and/or HMG-COA reductase) and a pharmaceutically acceptable carrier. In a further embodiment, a pharmaceutical composition can be provided comprising a therapeutically effective amount of at least one disclosed compound. In a still further embodiment, a pharmaceutical composition can be provided comprising a prophylactically effective amount of at least one disclosed compound. In yet a further embodiment, the disclosure relates to pharmaceutical compositions comprising a pharmaceutically acceptable carrier and a disclosed compound, wherein the compound is present in an effective amount. In an even further embodiment, the pharmaceutical compositions are useful in modulating NO neuron activity (e.g., inducing colonic motility). In a still further embodiment, the pharmaceutical compositions are useful in treating a gut motility disorder.

Thus, in various embodiments, provided herein are pharmaceutical compositions comprising a therapeutically effective amount of a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof, and a pharmaceutically acceptable carrier.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In further embodiments, the pharmaceutical composition is administered to a mammal. In still further embodiments, the mammal is a human. In an even further embodiment, the human is a patient.

In further embodiments, the pharmaceutical composition is administered following identification of the mammal in need of treatment of a disorder associated with NO neuron activity. In still further embodiments, the mammal has been diagnosed with a need for treatment of a disorder associated with NO neuron activity prior to the administering step.

In further embodiments, the pharmaceutical composition is administered following identification of the mammal in need of treatment of a gut motility disorder. In still further embodiments, the mammal has been diagnosed with a need for treatment of a gut motility disorder prior to the administering step.

In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyldialkylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, PA, Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment (e.g., prophylactic or therapeutic) of a gut motility disorder. The method also includes the administration of a therapeutically effect amount of the compound for the treatment of patient having a predisposition for being afflicted with a gut motility disorder. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder.

The total amount of the compound of the present disclosure administered in a typical treatment is preferably from about 1 mg/kg to about 100 mg/kg of body weight for mice, and from about 10 mg/kg to about 50 mg/kg of body weight, and from about 20 mg/kg to about 40 mg/kg of body weight for humans per daily dose. In some embodiments, the subject is a human and the dose is from about 10 mg/kg to about 90 mg/kg of body weight. In some embodiments, the subject is a human and the dose is from about 10 mg/kg to about 80 mg/kg of body weight. In some embodiments, the subject is a human and the dose is from about 10 mg/kg to about 70 mg/kg of body weight. In some embodiments, the subject is a human and the dose is from about 10 mg/kg to about 60 mg/kg of body weight. In some embodiments, the subject is a human and the dose is from about 1 mg/kg to about 300 mg/kg of body weight of the human. This total amount is typically, but not necessarily, administered as a series of smaller doses over a period of about one time per day to about three times per day for about 24 months, and over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In certain some embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

A compound described herein can be administered alone or can be coadministered with an additional therapeutic agent. Thus, the preparations can also be combined, when desired, with other active substances (e.g., to reduce metabolic degradation). Additional therapeutic agents include, but are not limited to, other active agents known to be useful in treating a gut motility disorder as further described herein.

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris (hydroxymethyl) amino methane HCl (pH7.0), 0.9% saline, 1.2% saline, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly (glycolide), poly (lactide), poly (epsilon-caprolactone), poly-(hydroxybutyrate) and poly (hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

It is understood that the disclosed compositions can be prepared from the disclosed compounds. It is also understood that the disclosed compositions can be employed in the disclosed methods of using.

### E. Kits

In some embodiments, disclosed are kits comprising a disclosed compound (e.g., modulators of COX, dopamine receptors, sodium channels, serotonin receptors, acetylcholine receptors, GABA receptors, FAAH, adrenergic receptors, histamine receptors, vasopressin receptors, NMDAR, beta amyloid, gamma-secretase, IxB/IKK, glutamate receptors, opioid receptors, TRPV, aldose reductase, calcium channels, glucocorticoid receptors, and/or HMG-COA reductase), or a pharmaceutically acceptable salt thereof, and one or more selected from: (a) an agent known for treating a gut motility disorder; (b) instructions for treating a gut motility disorder; and (c) instructions for administering the compound in connection with treating a gut motility disorder.

Thus, in various embodiments, disclosed are kits comprising a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof, and one or more selected from: (a) an agent known for treating a gut motility disorder; (b) instructions for treating a gut motility disorder; and (c) instructions for administering the compound in connection with treating a gut motility disorder.

In further embodiments, the kit comprises the agent known for the treatment of a gut motility disorder. Examples of agents known for the treatment of gut motility disorders include, but are not limited to, parasympathomimetics, prokinetic agents, opiod antagonists, antidarrheals, and antibiotics. In still further embodiments, the agent known for the treatment of a gut motility disorder is selected from neostigmine, bethanechol, metoclopramide, cisapride, and loperamide.

In further embodiments, the compound and the at least one agent are co-formulated. In further embodiments, the compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the disclosure to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

The enteric nervous system (ENS) plays a central role in gut physiology and mediating the crosstalk between the gastrointestinal (GI) tract and other organs. The human ENS has remained elusive, highlighting the need for an in vitro modeling and mapping blueprint. Here, an unbiased screen was conducted, and drug candidates were identified that modulate the activity of NO neurons. Next, their potential in promoting motility was demonstrated in mouse colonic tissue ex vivo. A high-throughput strategy was created to define the developmental programs involved in NO neuron specification, and it was discovered that PDGFR inhibition boosts the induction of NO neurons in enteric ganglioids. Transplantation of these ganglioids in the colon of NO neuron-deficient mice results in extensive tissue engraftment, providing a xenograft model for the study of human ENS in vivo and the development of cell-based therapies for neurodegenerative GI disorders. These studies provide a framework for deciphering fundamental features of the human ENS and designing effective strategies to treat enteric neuropathies.

Representative examples of the disclosed compounds are illustrated in the following non-limiting methods, schemes, and examples.

### 1. Methods

### A. Culture and Maintenance of Undifferentiated Human Stem Cells

Human embryonic stem cell (hESC) line H9 (WAe009-A, and reporter expressing derivatives hSYN::ChrR2-EYFP, NOS1::GFP) and induced pluripotent stem cell (hiPSC) line WTC-11 (UCSFi001-A) were plated on Geltrex™-coated plates and maintained in chemically-defined medium (E8) as described previously (Barber et al., 2019). The maintenance cultures were tested for mycoplasma every 30 days.

b. Enteric Neural Crest (ENC) Induction

When the monolayer cultures of hPSCs reached about 70% confluency, a previously established 12-day enteric neural crest (ENC) induction protocol was initiated (Barber et al., 2019; Fattahi et al., 2016) (D0) by aspirating the maintenance medium (E8) and replacing it with neural crest induction medium A [BMP4 (1 ng ml⁻¹), SB431542 (10 μM), and CHIR 99021 (600 nM) in Essential 6 medium]. Subsequently, on ENC induction days D2 and D4, neural crest induction medium B [SB431542 (10 μM) and CHIR 99021 (1.5 μM) in Essential 6 medium] and on D6, D8, and D10 medium C [medium B with retinoic acid (1 μM)] were fed to the cultures. Next, ENC crestospheres were formed during D12-D15 to facilitate the selection for ENC lineage and against contaminating ones in our cultures. In doing so, ENC induction crest medium C was removed on D12 and detached the ENC monolayers using accutase (30 min, 37° C., 5% CO2). After centrifuging the samples at 290×g for 1 min, the ENC cells were re-suspended in NC—C medium [FGF2 (10 ng ml⁻¹), CHIR 99021 (3 μM), N2 supplement (10 ul ml⁻¹), B27 supplement (20 ul ml⁻¹), glutagro (10 ul ml⁻¹), and MEM NEAAs (10 ul ml⁻¹) in neurobasal medium] and transferred to ultra-low-attachment plates to form free-floating 3D enteric crestospheres. On D14, when the free-floating enteric crestospheres could be observed, they were gently gathered in the center of each well using a swirling motion. Then, the old media was carefully aspirated from the circumference of each well without removing the crestospheres. After addition of the fresh NC—C medium, the cultures were incubated for 24 hours (37° C. and 5% CO2) prior to enteric neuron induction phase.

c. Enteric Neuron Induction from Enteric Neural Crests

On D15, enteric crestospheres were gathered in the center of the wells using a swirling motion and NC—C medium was removed using a P1000 micropipette in slow circular motion, avoiding the free-floating crestospheres. At this step protocol varied depending on the final desired culture layout (2D ENS cultures versus 3D enteric ganglioids). For 2D ENS cultures, after washing the enteric crestospheres with PBS, accutase (Stemcell Technologies, 07920) was added and plates were incubated for 30 minutes at 37° C. to dissociate the crestospheres. Then, remaining spheroids were broken by pipetting ENC medium [GDNF (10 ng ml⁻¹), ascorbic acid (100 μM), N2 supplement (10 μl ml⁻¹), B27 supplement (20 μl ml⁻¹), glutagro (10 μl ml⁻¹), and MEM NEAAs (10 μl ml⁻¹) in neurobasal medium]. Cells were spun (2 min, 290×g, 20-25° C.) and supernatant was removed. Pellet was resuspended in ENC medium and cells were plated on poly-L-ornithine (PO)/laminin/fibronectin (FN) plates at 100,000 viable cells per cm². For 3D enteric ganglioids, accutase treatment was avoided and enteric crestospheres were fed with the same volume of ENC medium [GDNF (10 ng ml⁻¹), ascorbic acid (100 μM), N2 supplement (10 μl ml⁻¹), B27 supplement (20 μl ml⁻¹), glutagro (10 μl ml⁻¹), and MEM NEAAs (10 μl ml⁻¹) in neurobasal medium]. Feeding continued every other day with ENC medium until D30-D40, after which, feeding frequency could be reduced to once or twice per week but with a larger volume of feeding medium.

d. Immunofluorescence

For immunofluorescence (IF) staining, cells were initially fixed in 4% PFA in PBS (30 min, room temperature (RT), and then blocked and permeabilized by permeabilization buffer (PB) (Foxp3/Transcription Factor Staining Buffer Set, 00-5523) for another 30 minutes at RT. After fixation and permeabilization steps, cells were incubated in primary antibody solution overnight at 4° C., and then washed three times with PB before their incubation with fluorophore-conjugated secondary antibodies at RT. Before imaging, stained cells were incubated with DAPI fluorescent nuclear stain and washed an additional three times. List of antibodies and working dilutions is not shown.

e. Preparation of Enteric Ganglioid Frozen Sections

hPSC-derived ganglioids were collected at stage 1 (day 37-50) and stage 2 (day 70-90), rinsed twice in PBS and fixed on ice in 4% PFA (SCBT sc-281692) for 3 hours, followed by replacing 90% of the supernatant with PBS for storage at 4° C. for up to 6 months. Ganglioids were treated with 5% sucrose (RPI Research Products 524060) in PBS for 10 minutes at room temp, followed by 10% sucrose in PBS for 2 hours at room temp and 20% sucrose at 4° C. overnight. Sucrose-treated ganglioids were positioned in cryomolds (Tissue-Tek® Cryomold® medium, VWR 25608-924), all 20% sucrose removed and incubated in 2:1 20% sucrose: OCT (Tissue Plus O.C.T. Compound Fisher HealthCare 5484) for 2 hours at room temperature before flash freezing in ethanol/dry ice. 1220 μm sections were taken on a cryostat (Leica 3050S) adhered to Superfrost® Plus Micro Slide, Premium (VWR 48311-703) and dried on 42° C. slide dryer for up to 2 hours before storing at −80° C. for up to a year.

f. Preparation of Paraffin-Embedded Human Colon Sections

Human sigmoid colon tissue was received from the International Institute for the Advancement of Medicine (IIAM) that provides non-transplantable organs from Organ Procurement Organizations for biomedical research purposes. Colon tissue was obtained under sterile conditions, flushed with isotonic solution, submerged in organ transplant solution, and shipped on ice to laboratory within 24 hours post mortem. Full-thickness tissues pieces (˜2 cm2) were fixed overnight (<24 hours) in 10% neutral buffered formalin (Cancer Diagnostics, FX1003). Samples were transferred to 70% ethanol prior to paraffin embedding (Leica ASP6025, tissue processor). Approximately 5 μM thick transverse tissue sections were cut onto coated glass slides (Superfrost® Plus Micro Slide; VWR, 48311-703) and air-dried overnight. All following slide preparation steps were performed at room temperature. Slides with paraffin sections were washed three times in clean xylene substitute (Sigma A5597), then once each in 100% ethanol, 95% ethanol, and 70% ethanol. Slides were then run under house DI water for 5 minutes before being placed in 1×PBS for storage at 4° C. for up to 4 weeks. Prior to staining, paraffin sections underwent antigen retrieval in either citrate buffer (Vector Laboratories Antigen Unmasking Solution H-3300) or TE buffer (Thermo 17890, brought to pH 9.0 with 1 M NaOH). Slides were incubated in buffer for 10 minutes at 95° C. using a Pelco BioWave Pro+ set to 400 watts.

g. Staining Enteric Ganglioid Frozen Sections and Paraffin-Embedded Human Colon Sections

Unless otherwise specified, all steps were performed at room temperature. Ganglioid frozen sections and paraffin-embedded human normal colon sections were prepared as above and then washed three times in PBS and blocked for 1-2 hours in serum (10% donkey or 10% goat) with 0.5% (v/v) Triton X-100 (VWR 0694). Slides were then incubated with primary antibody diluted in serum (10% donkey or 10% goat) with 0.1% Triton X-100 at 4° C. for 12-20 hours. Slides were washed six times for 20 minutes each in PBS with 0.1% Tween-20 (Sigma P1379) and incubated for 1 hour with Alexa Fluor conjugated secondary antibodies. The diluted secondary antibody solution was removed and replaced with 1.0 μg/mL DAPI in water for 10 minutes. The slides were washed six times for 20 minutes each in PBS with 0.1% Tween-20 and coverslips were mounted with Fluoromount-G (Southern Biotech 0100-01). List of antibodies and working dilutions is not shown. Images were acquired on a Leica SP8 inverted confocal or on the Echo Revolve. For images that were stitched Leica's LAS X tiling feature or the Grid/Pairwise stitching plugin for FIJI (PMID 19346324) were used.

### h. 2-Photon Fluorescence Imaging

Imaging experiments were conducted on a custom-built upright 2-photon microscope operating with μManager software (San Francisco, CA). The excitation source was a 2-photon Coherent Chameleon Vision II laser operating at 760 nm (Coherent, Santa Clara, CA). Images were collected using an Olympus LWD 1.05 NA water immersion objective (Olympus, Tokyo Japan). An emission filter collecting light between 380 nm-420 nm (Chroma, Bellow Falls VT) were used to image DAPI, while the fluorescence emission of Alexa 568 was collected using a filter between 565 nm and 635 nm (Chroma, Bellow Falls VT).

i. Macro Fluorescence Imaging

Images were taken on a Nikon AZ100M “Macro” laser scanning confocal configured with long working distance low magnification lenses. The microscope is equipped with the standard 405 nm, 488 nm, 561 nm, and 640 nm laser lines and has PMT detectors with a detection range from 400-700 nm. To reduce signal drop-off at the image edges an optical zoom factor of 2.1x was used, and the lateral resolution was increased using a digital zoom factor of 1.873x.

j. Flow Cytometry

For preparation of samples for flow cytometry analysis, cells were initially dissociated into single cell suspensions by accutase treatment (Stemcell Technologies, 07920, 30-60 min, 37° C., 5% CO2) and then fixed and permeabilized using fixation/permeabilization buffers (Foxp3/Transcription Factor Staining Buffer Set, 00-5523). Cells were stained with primary and secondary antibodies as described above for immunofluorescence. Flow cytometry was conducted using a BD LSRFortessa cell analyzer and data were analyzed using Flowjo™ (FlowJo™ Software Version 8.7). List of antibodies and working dilutions is not shown.

k. Human Synapsin::Channelrhodopsin2-EYFP Enteric Ganglioids Blue Light Activation

Enteric ganglioids were either exposed to blue light (100% laser intensity, 3×1-min exposure with 30 s intervals, EVOS FL) or left out in ambient light. Enteric ganglioids were then incubated for 45 minutes at 37° C. before dissociation, fixation and permeabilization for flow cytometry (see above). Cells were stained using antibodies against cFos (abcam, ab190289) and TUBB3 (Biolegend, 801202).

### 1. Bulk RNA-Seq Data Analysis

Total RNA was extracted using PureLink™ RNA Mini Kit. First strand cDNA was then synthesized with the Quantseq Forward Library preparation kit from Lexogen. Illumina compatible RNA sequencing libraries were prepared with Quantseq and pooled and sequenced on Illumina Hiseq 4000 platform at the UCSF Center for Advanced Technology. UMIs were extracted from the fastq files with umi_tools, and cutadapt was used to remove short and low-quality reads. The reads were aligned to human GENCODE v.34 reference genome using STAR aligner, and the duplicate reads were collapsed using umi_tools. Gene level counts were measured using HTSeq and compared using DESeq2.

m. Single Cell and Single Nuclei RNA Sequencing Sample Preparation and Data Collection

All tubes and pipet tips used for cell harvesting were pre-treated with 1% BSA in 1×PBS. Cells were dissociated in Accutase (Stem Cell) at 37° C., in 10 min increments, with end-to-end rotation, until single cell suspension was obtained. The cells were washed in Cell Staining Buffer (Biolegend) and stained with TotalSeq HTO antibodies for 30 min on ice. The cells were washed twice in Cell Staining Buffer and filtered through a 40 μm pipette tip strainer (BelArt). The cells were counted using Trypan Blue dye and hemocytometer and pooled for sequencing. scRNA-seq libraries were prepared with Chromium Next GEM Single Cell 3′ Kit v3.1 (10× Genomics), with custom amplification of TotalSeq HTO sequences (Biolegend). The libraries were sequenced on Illumina NovaSeq sequencer in the Center for Advanced Technologies (UCSF). The cell feature matrices were extracted using kallisto/bustools, and demultiplexed using seurat.

n. Quality Control and Cell Filtration

Datasets were analyzed in R v4.0.3 with Seurat v4 (Hao et al., 2021). The number of reads mapping to mitochondrial and ribosomal gene transcripts per cell were calculated using the “PercentageFeatureSet” function. Cells were identified as poor quality and subsequently removed independently for each dataset based on the number of unique features captured per cell, the number of UMI captured per cell and the percentage of reads mapping to mitochondrial transcripts per cell. Dataset specific quality control metric cutoffs are not shown.

o. Dimensionality Reduction, Clustering and Annotation

Where applicable, biological replicate samples were first merged using the base R “merge” function. Counts matrices were log normalized with a scaling factor of 10,000 and 2,000 variable features were identified using the “vst” method. Count matrices of biological replicate samples were integrated using Seurat integration functions with default parameters. Cell cycle phase was predicted using the “CellCycleScoring” function with Seurat's S and G2M features provided in “cc.genes.” The variable feature sets were scaled and centered, and the following variables were regressed out: nFeatures, nCounts, mitochondrial gene percentage, ribosomal gene percentage, S score and G2M score. Principal Components Analysis (PCA) was run using default settings and Uniform Manifold Approximation and Projection (UMAP) dimensionality reduction was performed using the PCA reduction. The shared nearest neighbors (SNN) graph was computed using default settings and cell clustering was performed using the default Louvain algorithm. Quality control metrics were visualized per cluster to identify and remove clusters of low-quality cells (less than average nFeatures or nCounts and higher than average mitochondrial and ribosomal gene percentage). The above pipeline was performed again on datasets after the removal of any low-quality cell clusters and for the sub-clustering analysis of the enteric neural crest, enteric neurons, nitrergic neurons and enteric glia. The number of principal components used for UMAP reduction and SNN calculation was determined by principal component standard deviation and varied for each dataset. The number of principal components used for SNN and UMAP calculation and the resolution used for clustering of each dataset is not shown. Cluster markers were found using the Wilcoxon Rank Sum test and clusters were annotated based on the expression of known cell type marker genes. Following cell type annotation, gene dropout values were imputed using adaptively-thresholded low rank approximation (ALRA) (Linderman et al., 2018). The rank-k approximation was automatically chosen for each dataset and all other parameters were set as the default values. The imputed gene expression is shown in all plots and used in all downstream analysis unless otherwise specified.

p. Analysis of Published Datasets

Quality control. Criteria used by the original authors of each dataset was used to identify and remove poor quality cells. Dataset specific quality control metric cutoffs are not shown.

Dimensionality Reduction and Clustering. Datasets were analyzed with Seurat using the methods and parameters described by the original authors.

Morarach et al.: For all datasets, count matrices were normalized, mitochondrial gene percentage was regressed and 3000 variable features were returned using the “SCTransform” function. Highly expressed sex-specific and immediate early genes (Xist, Gm13305, Tsix, Eif253y, Ddx3y, Uty, Fos, Jun, Junb, Egr1) were removed form the variable feature list prior to running PCA. The dataset specific parameters used for the “RunUMAP”, “FindNeighbors” and “FindClusters” functions are not shown. Cell annotations determined by the authors were used for cell types and neuronal subtypes.

Drokhlyansky et al.: For all datasets, count matrices were log normalized with a scaling factor of 10,000 and 2,000 variable features were identified using the “vst” method. Batch correction by “Unique_ID” was performed using mutual nearest neighbors correction (MNN) with the “RunFastMNN” Seurat Wrappers function. The dataset specific parameters used for the “RunUMAP”, “FindNeighbors” and “FindClusters” functions are not shown. Cell annotations determined by the authors were used for cell types and neuronal subtypes. For consistency of comparison, gene dropout values were imputed using ALRA for all published datasets using automatically determined rank-k approximations and all other default values. The imputed gene expression is shown in all plots and used in all downstream analysis unless otherwise specified.

Glia Sub-clustering analysis. Glia were sub-clustered using methods similar to the original analysis pipeline described by each author above.

Morarach et al.: The E18 dataset contained a single transcriptionally homogenous glia cluster, so the glia and progenitor populations were sub-clustered together to provide comparative cell populations needed for downstream analysis. Subset datasets were then normalized, mitochondrial gene percentage was regressed and 3000 variable features were returned using the “SCTransform” function. Highly expressed sex-specific and immediate early genes (Xist, Gm13305, Tsix, Eif253y, Ddx3y, Uty, Fos, Jun, Junb, Egr1) were removed form the variable feature list prior to running PCA. The dataset specific parameters used for the “RunUMAP”, “FindNeighbors” and “FindClusters” functions are not shown.

Drokhlyansky et al.: Glia subset datasets were log normalized with a scaling factor of 10,000 and 2,000 variable features were identified using the “vst” method. Batch correction by “Unique_ID” was performed using mutual nearest neighbors correction (MNN) with the “RunFastMNN” Seurat Wrappers function. The dataset specific parameters used for the “RunUMAP”, “FindNeighbors” and “FindClusters” functions are not shown.

q. Gene Group Expression Characterization

Gene lists were compiled for genes belonging to ten different functional groups (transcription factors, neurotransmitter synthesis, neuropeptides, neurotransmitter receptors, neuropeptide receptors, cytokines, cytokine receptors, secreted signaling ligands, ligand receptors, and surface markers). For each dataset, the gene lists were filtered to remove low abundance genes (detected in less than 25% of cells of each cluster). Genes from these lists were determined to be exclusively expressed by a cluster if greater than 25% of cells of only a single cluster expressed the gene.

r. Cell Type Transcriptional Signature Scoring

To find transcriptionally similar cell populations between two datasets, first the differentially expressed (DE) genes of the reference dataset are calculated from the non-imputed gene counts with the “FindAllMarkers” function using the Wilcoxon Rank Sum test and only genes with a positive fold change were returned. The DE gene lists are first filtered to remove genes not present in the query dataset. Then for each cell cluster in the reference dataset, a transcriptional signature gene list is made from the top 100 DE genes sorted by increasing adjusted p-value. The query dataset is then scored for the transcriptional signature gene lists of each reference dataset cell cluster using the “AddModuleScore” function based on the query dataset's imputed gene counts.

### S. Spearman Correlations

The transcriptional correlation of cell clusters in two datasets was calculated from the non-imputed gene counts and utilized Seurat's integration functions to first find 3,000 anchor features based on the first 30 dimensions of the canonical correlation analysis and then integrate the two datasets using the same number of dimensions. The expression of these 3000 anchor features was then scaled and centered in the merged data object and the average scaled expression of each anchor feature was calculated for each dataset's cell clusters of interest using the “AverageExpression” function. A Spearman correlation matrix comparing all cell clusters to all cell clusters was generated based on the average scaled expression of the 3000 anchor features.

t. SWNE Projections

The reference and query dataset counts matrices are first filtered to only include genes detected in both datasets. Similarly Weighted Nonnegative Embeddings (SWNE) are then generated for the reference dataset using the SWNE v0.6 package. First, nonnegative matrix factorization (NFM) generates component factors from the 3000 variable features calculated from the reference dataset non-imputed gene counts. Two dimensional component factor embeddings are calculated using sammon mapping and the cells and specified key genes are embedded in 2D relative to the component factors. Finally, a SNN network is calculated from the reference dataset and is used to smooth the cell positions. The query dataset is then mapped onto the reference dataset's 2D component factor space by first projecting the query dataset onto the reference dataset's NFM factors. The resulting query dataset cell embeddings are then smoothed by projection onto the reference dataset's SNN network.

u. Myenteric and Submucosal Scoring

Patient metadata published by the authors was used to separately group neurons or glia by tissue layer origin. Pan-neuronal and pan-glial myenteric and submucosal gene signatures were created by performing the Wilcoxon Rank Sum test to identify DE genes between the myenteric and submucosal cell groupings. Neuronal and glial datasets were scored with the cell-type specific tissue layer signatures by first ordering the gene lists by increasing adjusted p-value and removing genes not detected in the dataset to be scored. The “AddModuleScore” function was then used to score the cells for the 100 most significantly enriched genes for each tissue layer.

v. Neurochemical Identification of Neurons

The neurochemical identification of neurons was performed independently for each neurotransmitter to accommodate multi-neurochemical identities. For each neurotransmitter, a core set of genes were selected consisting of the rate-limiting synthesis enzyme(s), metabolism enzymes and transport proteins. Cells were first scored for each neurotransmission associated gene set using the “AddModuleScore” function. A cell was then annotated as “x-ergic” if the cell's expression of a rate limiting enzyme was greater than 0 and the cell's module score for the corresponding gene set was greater than 0. A cell was annotated as “Other” if both criteria were not met. Multi-neurochemical identities were determined by concatenating the individually determined single neurochemical identities of each cell. The overall prevalence of each neurochemical identity per dataset was calculated by summing the total number of cells annotated for each single identity and calculating the percentage of each “x-ergic” identity from this sum total.

w. Neurotransmitter Response Scoring

Separate gene lists were created containing all receptors activated by each neurotransmitter. Cells were scored for their expression of each neurotransmitter receptor family gene set using the “AddModuleScore” function.

### X. Glia Gene Set Enrichment Analysis (GSEA) Hierarchical Clustering

For each sub-clustered glia dataset, DE genes for each glial subtype were calculated using the “FindAllMarkers” function. GSEA for the MSigDB gene ontology sets was performed on each glia subytpe's upregulated DE genes (positive log 2 fold change only) sorted by decreasing log 2 fold change using fgsea v1.16. Normalized Enrichment Scores (NES) were calculated for gene sets containing a minimum of 15 genes in the DE gene list with the scoreType set to “positive”. Each glial subtype's GSEA results were filtered to only include biological process gene sets but not filtered based on significance as to not limit the result to pathways enriched in the highest fold change genes. The NES of the filtered GSEA results for all glial subtypes were then merged and pathways not detected in a glial subtype were assigned a NES of 0. Hierarchical clustering was then performed based on the NESs to cluster both the gene ontology pathways and the glial subtypes. After glia classes were determined by clustering, pathways enriched in each class were identified by filtering for pathways with an NES greater than 1.1 in all subtypes of a given class.

y. PP121 Vs Control Gene Expression Correlation

To compare the gene expression of control and PP121 treated cell types, neuronal subtypes and NO neuron subtypes, a subset dataset of each cell type and subtype annotation was first created. For each subset, the non-imputed average expression of all genes was then calculated for the control and PP121 treated cells using the “AverageExpression” function and natural log transformed for plotting. R2 values comparing the control and PP121 natural log expression values were calculated from linear modeling using the “y˜x” formula.

z. cFOS Expression Screening

Stage 2 enteric ganglioids were dissociated using accutase and single cell suspensions (in ENC medium) were distributed in wells of V-bottom 96-well plates. Compounds from a neuronal signaling compound library (Selleckchem, USA) were added at 1 pM using a pin tool and cells were incubated for 75 minutes at 37° C. Afterwards, cells were washed with PBS, and were immediately fixed for flow cytometry.

aa. NO Release Assay

For high-throughput measures of nitric oxide (NO) release, stage 1 2D ENS cultures (96-well plates) were used. After washing cells with Tyrode's solution [NaCl (129 mM), KCl (5 mM), CaCl2 (2 mM), MgCl (1 mM), glucose (30 mM) and HEPES (25 mM) at pH 7.4], 70 pl/well of Tyrode's solution was added to each well. Neuronal signaling compounds (Selleckchem, USA) were added at 1 pM using a pin tool. After a 45 minutes incubation at 37° C., supernatants were used to determine NO release using an NO assay kit (Invitrogen, EMSNO). Briefly, the kit uses the enzyme nitrate reductase that converts nitrate to nitrite which is then detected as a colored azo dye absorbing light at 540 nm. NO release for each compound was presented as the A540 nm relative to the vehicle (DMSO).

Bb. High-Throughput Screening to Identify Compounds that Enrich NO Neurons

Day 15 H9 hESC-derived enteric crestospheres were dissociated into single cells (accutase, Stemcell Technologies, 07920, 30 min, 37° C.), resuspended in ENC medium and were transferred into 384-well plates. Plates were incubated for 2 hours for cells to attach. Using a pin tool, drugs from a library of 1694 inhibitors (SelleckChem, USA) were added to wells at the final concentration of 1 pM and plates were incubated with drugs until D20, when media were changed to ENC with no drugs. At day 40, cells were fixed, stained for NOS1 and imaged using InCellAnalyzer 2000 (GE Healthcare, USA). Hits were selected based on the fold increase of the percentage of NOS1⁺ cells relative to the wells treated with vehicle (DMSO).

cc. Surface Marker Screening

For human surface marker screening, PP121-treated NOS1::GFP enteric ganglioids from four independent differentiations were pooled, dissociated into single cells (accutase, Stemcell Technologies, 07920, 30-60 min, 37° C., 5% CO2) and fixed (Foxp3/Transcription Factor Staining Buffer Set, 00-5523, 30 min, 4° C.). Cells were permeabilized and blocked (same staining kit) prior to incubation with anti GFP antibody (abcam, ab13970, 4° C.). After three washes, cells were stained with Alexa Fluor 488-conjugated secondary antibody (40 min, RT). Secondary antibody solution was removed (3× washes) and cells were incubated with a blocking buffer containing PBS and 2% FBS (30 min, on ice). Cells were divided in a 240:16 ratio corresponding to the number of library antibodies raised in mouse and rat, and received anti-mouse and anti-rat Alexa Fluor 647-conjugated secondary antibodies respectively. Then, they were distributed into V-bottom 96-well plates and treated with library antibodies for 30 minutes on ice (BD Biosciences, 560747). After two washes, surface marker and GFP signals were quantified by high-throughput flow cytometry (BD LSRFortessa). NO neuron specific surface markers were identified based on the highest sensitivity (highest percentage of CD⁺GFP⁺ cells) and highest specificity (lowest percentage of CD⁺GFP-cells).

dd. Drug Target Interaction Prediction

Canonical SMILES of the hits were obtained from PubChem (De Giorgio et al . . . 2016; Niesler et al . . . 2021), and a list of their known and predicted targets was generated by combining data from the following databases: BindingDB (https://www.bindingdb.org/), Carlsbad (http://carlsbad.health.unm.edu/),

DINIES (https://www.genome.jp/tools/dinies/), PubChem BioAssay (https://pubchem.ncbi.nlm.nih.gov/, filtered for active interactions), SEA (http://sea.bkslab.org/, filtered for MaxTC>0.4), SuperDRUG2 (http://cheminfo.charite.de/superdrug2/) and SwissTargetPrediction (http://www.swisstargetprediction.ch/).

ee. In Vivo Cell Transplantation

Specified pathogen free (SPF) homozygote neuronal nitric oxide synthase knockout mice (B6.129S4-Nos1^{tm1Plh}/J; nNos1^{-/-}) were bred and maintained, in individually ventilated cages (IVC), for use as recipients. Animals used for these studies were maintained, and the experiments performed, in accordance with the UK Animals (Scientific Procedures) Act 1986 and approved by the University College London Biological Services Ethical Review Process. Animal husbandry at UCL Biological Services was in accordance with the UK Home Office Certificate of Designation. As Nos1^{-/-} mice are immunocompetent, cyclosporin A (250 μg/ml in drinking water) was administered orally two days prior to transplantation to reduce the possible rejection of donor human cells. Cyclosporin A-treated Nos1^{-/-} mice were chosen at random, from within littermate groups, and stage 1 enteric ganglioids were transplanted into the of P23-P27 mice, via laparotomy under isoflurane anesthetic. Briefly, the distal colon was exposed and enteric ganglioids, containing 0.5-1 M cells were subsequently transplanted to the serosal surface of the distal colon, by mouth pipette, using a pulled glass micropipette. Each transplanted tissue typically received 3 ganglioids which were manipulated on the surface of the distal colon, with the bevel of a 30 G needle, to ensure appropriate positioning. Transplanted Nos1^{-/-} mice were maintained with continued free access to cyclosporin A (250 jig/ml) treated drinking water for up to 8 weeks post-transplantation, to ensure extended immunosuppression, before sacrifice and removal of the colon for analysis. As cyclosporin A can affect several signaling pathways and induce gene expression changes, it is crucial to verify immunofluorescence results using appropriate controls such as tissue from cyclosporin A treated untransplanted animals in follow up studies. In addition, other immunocompromised backgrounds (e.g., NSG) will be important to further verify these engraftment results.

ff. Tissue Preparation and Fixation

Following the excision, the entire colon was pinned in a Sylgard (Dow, MI, USA) lined petri dish and opened along the mesenteric border. The mucosa was subsequently removed by sharp dissection and tissues were fixed in 4% PFA in PBS (45 min-1 hour, 22° C.) for further processing.

gg. Tissue Staining

Colonic longitudinal muscle myenteric plexus (LMMP) tissues were fixed with 4% PFA (1 h on ice), Thermo scientific, J19943-K2) and blocked and permeabilized with a buffer containing 1% BSA and 1% triton X-100 (in PBS, 45 min, RT). Then, tissues were incubated with primary antibody solutions (in the same buffer, overnight, 4° C.) and were washed three times before treatment with fluorophore-conjugated secondary antibodies (1 h, RT). Samples were stained with DAPI and washed prior to mounting using vectashield (Vector Laboratories, H-1400). List of antibodies is not shown.

hh. Multi-Electrode Array (MEA) Analysis

Data acquisition: Neuron activity was recorded with the Axion Maestro Edge on Cytoview MEA 24-well plates in 1-hour recording sessions for each condition. Neuormodulator or vehicle were added by removing the plate from the Maestro Edge, half-changing the media with 2× concentrated neuromodulator or vehicle in pre-warmed media, and immediately returning the plate to the Axion to resume recording. Optogenetic stimulation was performed with the Axion Lumos attachment, stimulating all wells of the plate with 488 nm light at 50% intensity, 1 second on, 4 seconds off, 30 times.

Data processing: Raw data were first spike sorted with a modified version of SpikeInterface (https://github.com/SpikeInterface) using MountainSort to identify high quality units by manually scoring based on amplitude, waveform shape, firing rate, and inter-spike interval contamination. For pharmacology experiments, neurons were matched between vehicle and neuromodulator recordings by examining all detected units on a specific electrode after spike scoring and identifying units with identical waveforms. Firing rates of these “paired” units from all wells that received the treatment were compared across the control and neuromodulator conditions. Positive responders were units that had a firing rate change greater than +0.1 Hz; negative responders had a firing rate change less than −0.1 Hz; neutral responders had a firing rate change between −0.1 to +0.1 Hz. For optogenetic experiments, individual units were again extracted with SpikeInterface and manually scored. Recordings were separated into “on” times when the LED was active and “off” times when it was not. All units were compiled and firing rates for each unit were compared during the on and off windows.

ii. Ex Vivo Colonic Motility Assays

Preparation of solutions: Krebs buffer [NaCl (117 mM), KCl (4.7 mM), NaH₂PO₄ (1.2 mM), MgCl₂ (1.5 mM), CaCl₂·2H₂O (2.5 mM), NaHCO₃ (25 mM), Glucose (11 mM), pH 7.4] was placed in a 37° C. water bath and aerated with 95% 02 and 5% CO2 (carbogen) gas mixture for at least 30 minutes prior to experiment onset. “Drug” treatment solutions were freshly prepared by adding the drug compound into Krebs buffer before starting data acquisition. The solution with NOS1 inhibitor was prepared by adding N omega-nitro-L-arginine methyl ester hydrochloride (L-NAME) to the drug solutions making “Drug+L-NAME”.

Tissue dissection: For each experimental replicate, a pair of 8-week-old wild type C57BL6 mice (male) were placed in a sealed chamber and euthanized using CO2 asphyxiation followed by cervical dislocation. The lower GI tract (cecum and colon) was removed and immediately transferred to 37° C. carbogenated Krebs buffer, with the fecal matter still inside. Adipose tissue and mesentery were removed before placing the colons in the organ bath reservoir of gastrointestinal motility monitor (GIMM) apparatus. GIMM had two reservoirs making simultaneous acquisition of control, and drug-treated colons possible.

Experimental set-up and procedure: GIMM was designed based on a previously reported model (Swaminathan et al., 2016). The organ reservoir of GIMM has two-chambers for recording two specimens simultaneously. It is connected to working solutions kept at 37° C. via a 4-channel peristaltic pump (WPI, PERIPRO-4LS). Lower GI tract was harvested and transferred to the organ bath with the Krebs buffer was flowing through. The cecum was pinned down at the proximal tip and the distal end of the colon was pinned through the serosa/mesentery. Five 10-min (for the first experiments) or sequential 6-min (for the sequential drug treatment in the presence and absence of L-NAME) videos were recorded using the IC capture software (Imaging Source) with a high resolution monochromatic firewire industrial camera (Imaging Source®, DMK41AF02) connected to a ⅔″ 16 mm f/1.4 C-Mount Fixed Focal Lens (Fujinon HF16SA1). While tissue in the control chamber was only exposed to Krebs solution, the order of solutions in the experimental chamber was: Krebs, drug compound, Krebs (6 min each), L-NAME (2 min), L-NAME in the presence of a drug compound (6 min) and Krebs (6 min). The chambers were cleaned after each acquisition.

Data and statistical analysis: VolumetryG9a was used to generate the spatiotemporal map (STM) of each acquisition (Spear et al., 2018). Slow waves (SW) and colonic migrating motor complexes (CMMC) data were generated from STMs. Statistical analyses were performed using PRISM.

2. hPSC-Derived ENS Models Identify Modulators of No Neurons that Promote Colonic Motility

Given the significant role of enteric NO neurons in GI motility and their selective vulnerability in a wide range of congenital and acquired enteric neuropathies (Bódi et al., 2019; Rivera et al., 2011), there has been a great interest in establishing strategies to regulate their function. Factors that modulate NO neuron activity and increase NO release will facilitate the identification of potential drug targets for treatment of enteric neuropathies. Hence, our scalable ENS culture platforms were leveraged to screen for compounds that induce NO neuron activity.

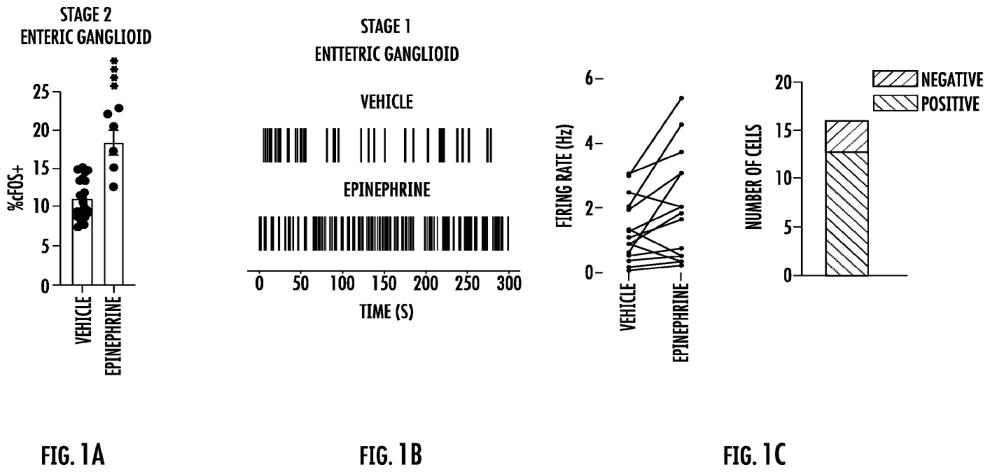

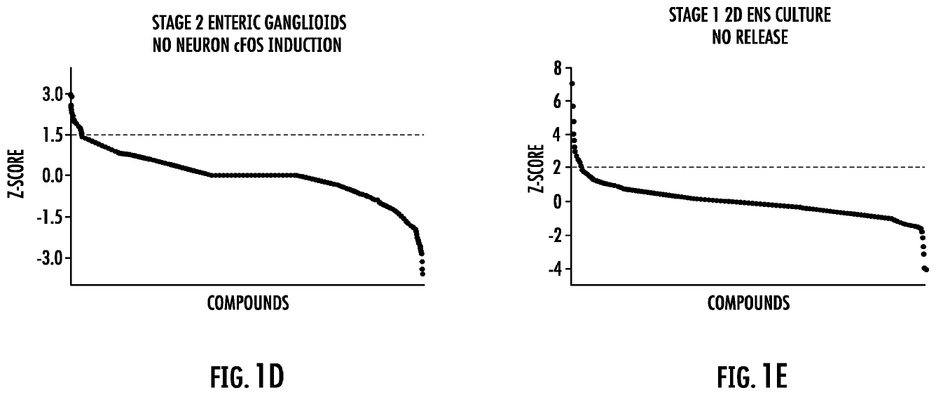

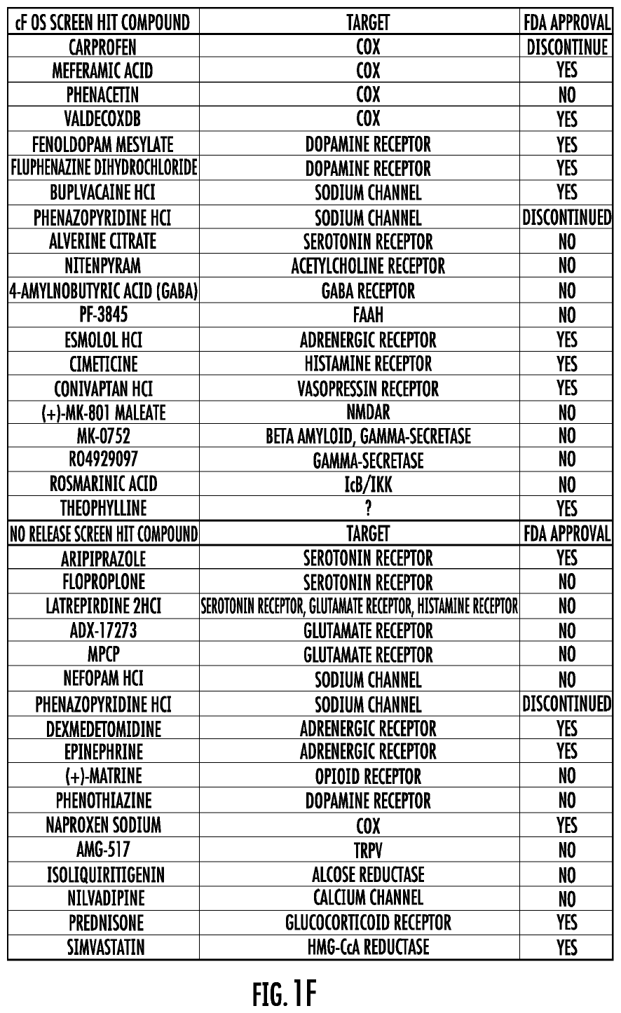

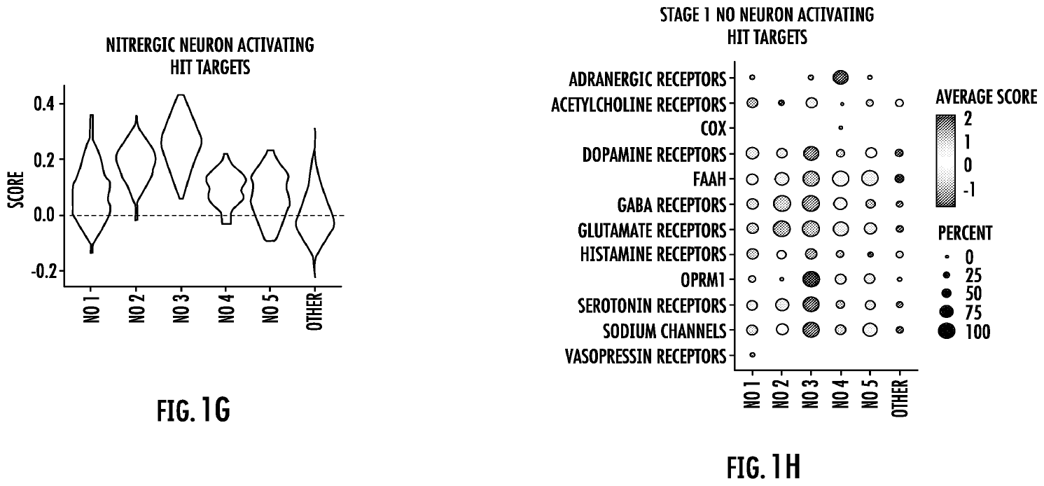

A screening strategy for NO neuron activity was developed based on the induction of cFOS expression as a readout. In order to evaluate cFOS as an accurate read-out of neurochemical-induced activity, side by side cFOS flow cytometry analysis and MEA neuronal firing measurements were performed in cultures treated with epinephrine, which is known to stimulate enteric neurons. Epinephrine induced neuronal cFOS expression and resulted in increased electrical firing of ganglioid neurons. This provides a scalable read-out of activity that is suitable for high-throughput screens. (FIG. 1A-C).

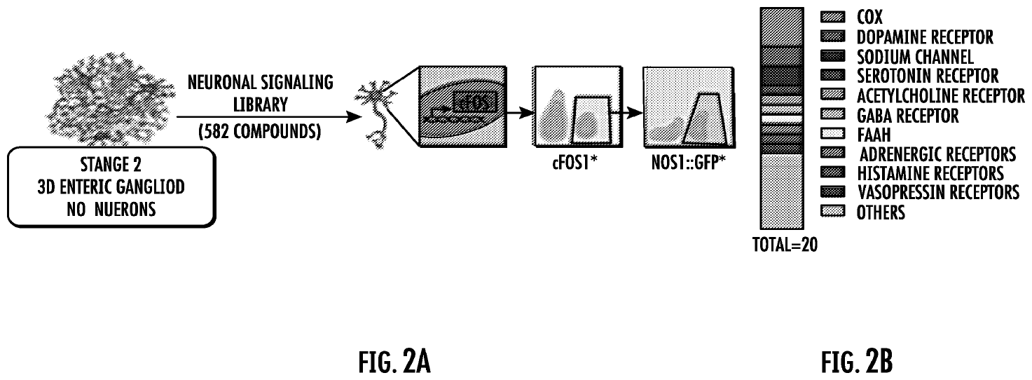

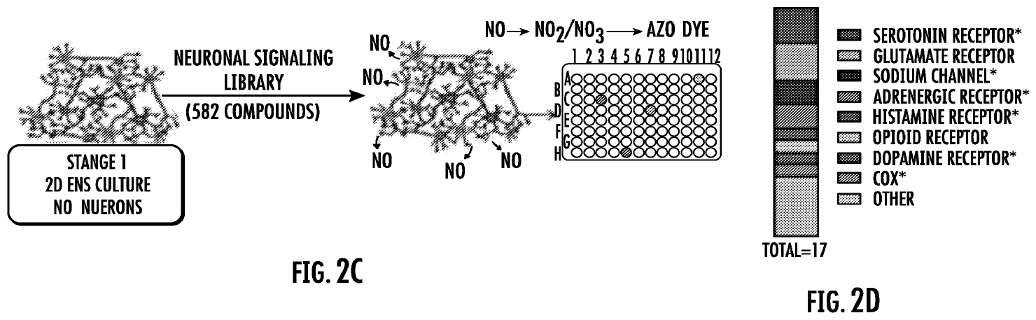

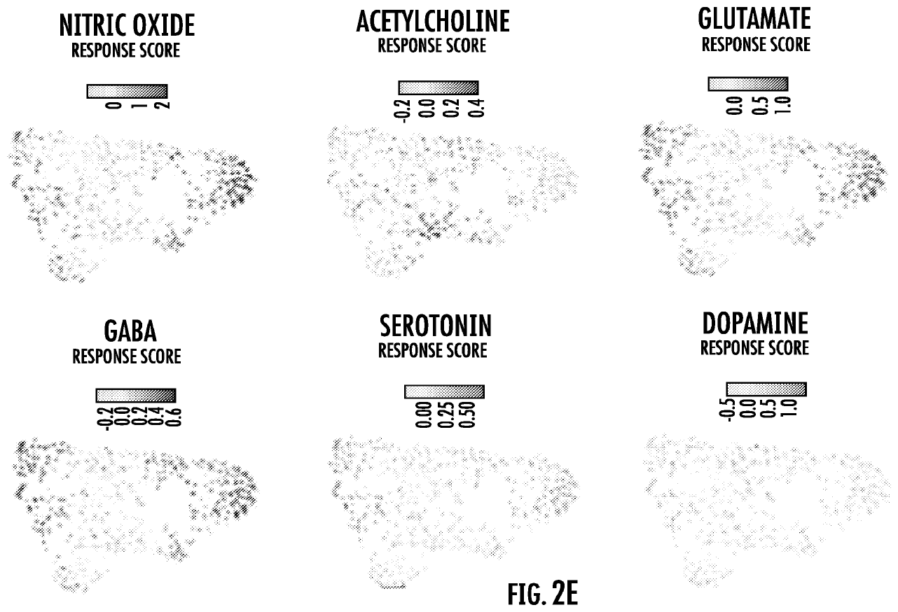

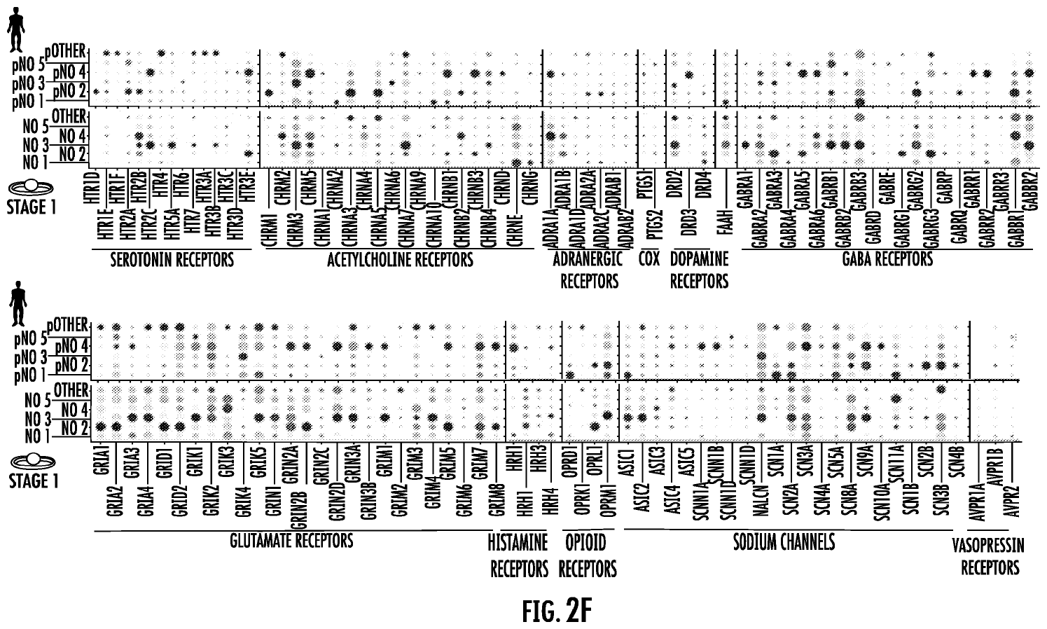

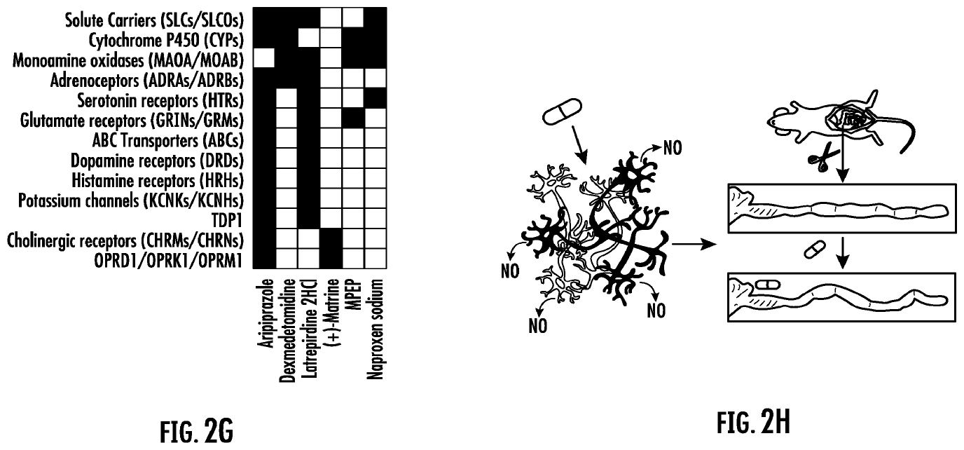

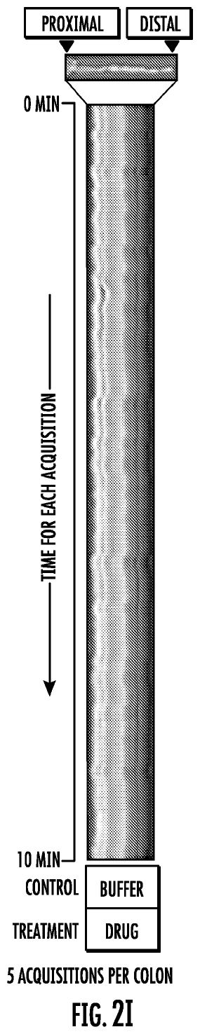

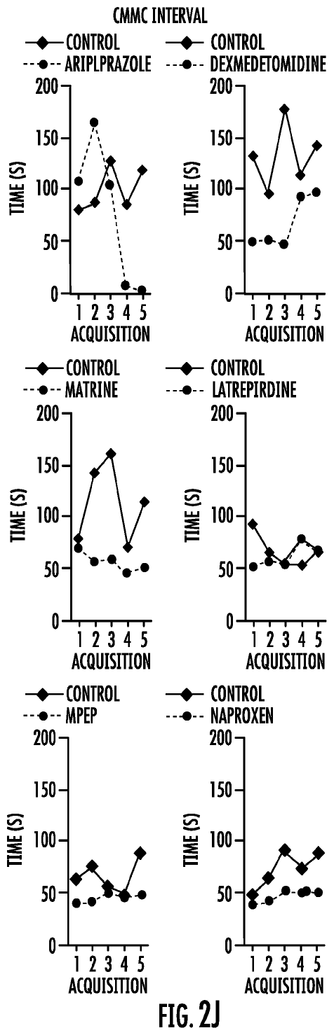

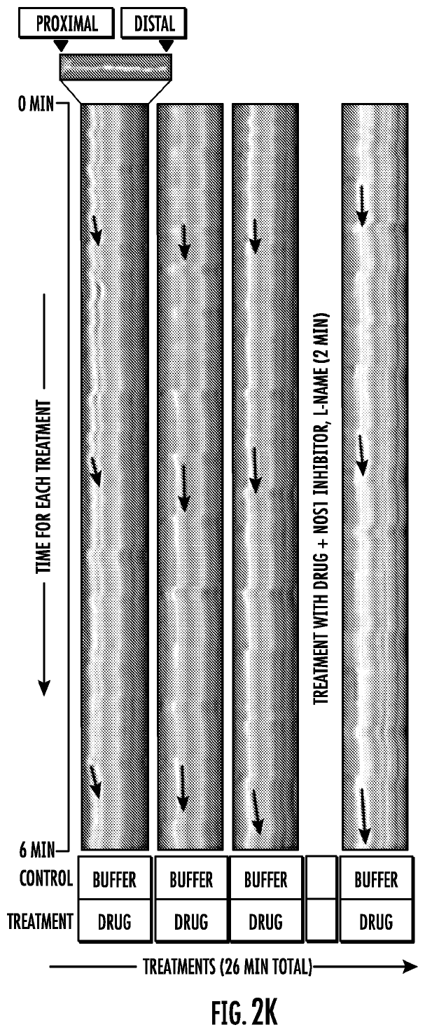

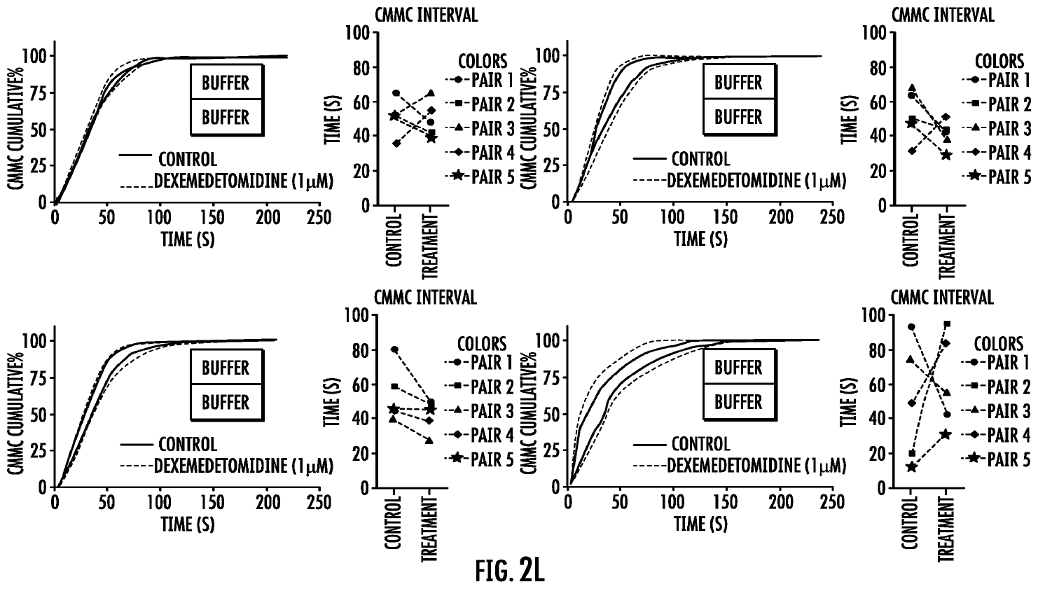

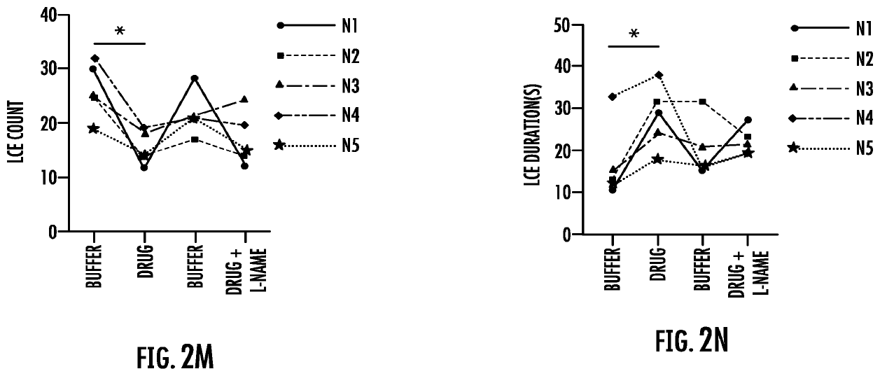

First, a cFos induction screen was performed, where NOS1::GFP enteric ganglioid cells were exposed to a library of 582 neuromodulators (Selleck neuronal signaling Library™) and co-expression of cFOS and GFP was measured to quantify NO neuron activity (FIG. 2A and FIG. 1D). 20 compounds were identified that increased the proportion of NO neurons in cFOS⁺ cells with z-score>1.5. To identify the mechanisms involved in cFOS expression in NO neurons, the list of target proteins was compiled and classified, and multiple shared protein classes were discovered. In particular, these proteins converged on serotonin receptors, sodium channels, acetylcholine receptors, glutamate receptors, adrenergic receptors, histamine and opioid receptors and dopamine receptors (FIG. 2B).

In an independent functional screen, a high-throughput read-out for assessing NO neuron activity was established. A commercially available kit was utilized, which enables NO detection in the media. Upon release into the media, NO is spontaneously oxidized to nitrate. The kit uses nitrate reductase to convert nitrate to nitrite that is then detected as a colored azo dye. 2D ENS cultures were incubated with the neuromodulators library and measured NO release using calorimetry (FIG. 2C). 17 compounds were identified that remarkably enhanced NO concentration in the supernatant with z-score>2.0 (FIG. 1E). Neuromodulators that induced NO release in the ENS cultures were diverse but were predicted to commonly target protein classes including serotonin receptors, sodium channels, acetylcholine receptors, glutamate receptors, adrenergic receptors and opioid receptors (FIG. 2D).

Interestingly, there was a high degree of similarity between the predicted targets from the cFOS induction and NO release screens (FIG. 2B, FIG. 2D, and FIG. 1F). These targets included receptors for neurotransmitters such as serotonin and dopamine. Module scoring the neurotransmitter receptor gene families in the hPSC-derived stage 1 enteric NO neurons snRNA-seq data confirmed that NO neurons broadly express receptors for NO, serotonin, GABA, glutamate, acetylcholine or dopamine (FIG. 2E). Interestingly, compared to other neuronal subtypes, stage 1 NO neuron clusters were enriched for all predicted hit targets (FIG. 1G). In particular, NO 3 cluster scored high for the expression of the majority of NO neuron modulator target classes (FIG. 1H). Profiling the expression of individual genes in each target protein category in ganglioids and primary human ENS revealed notable subtype-specific expression patterns between NO neurons. For example, GABA receptor genes were predominantly expressed by NO 2, NO 3, and pNO 4 subtypes, while the expression of acetylcholine receptor genes was less specific to a particular subtype (FIG. 2F).

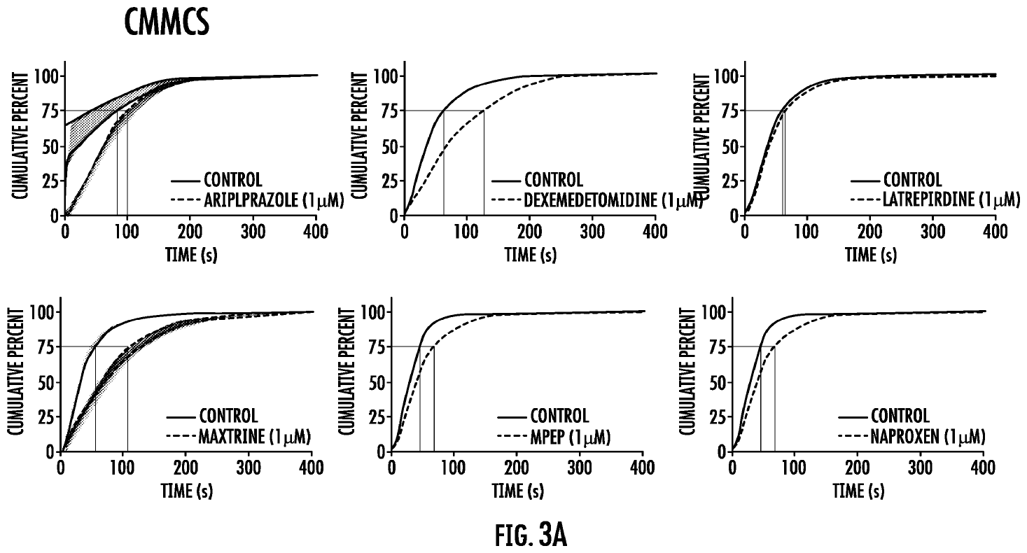

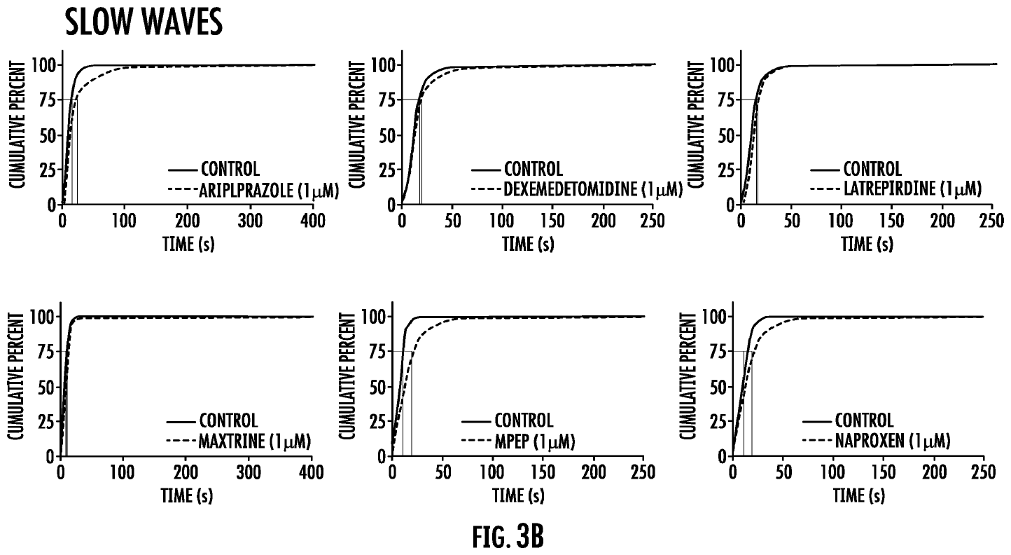

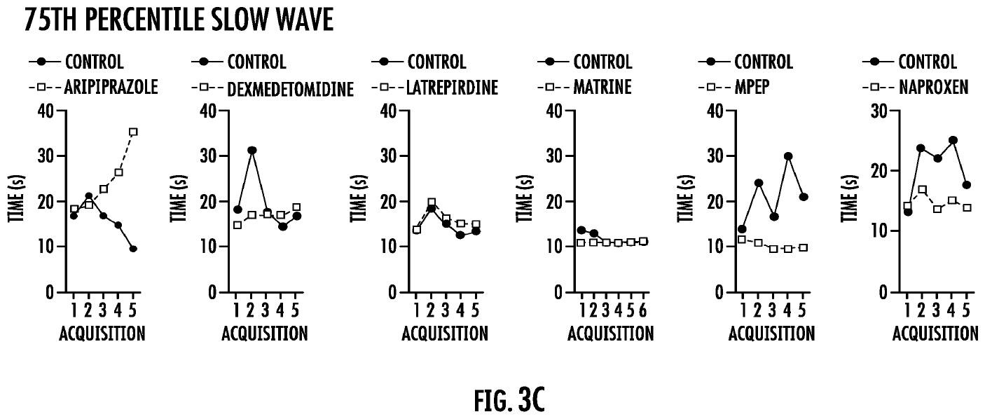

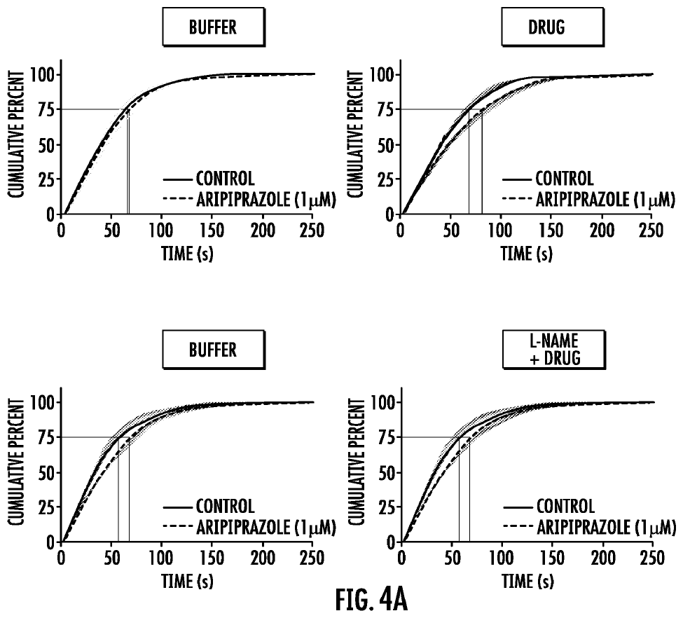

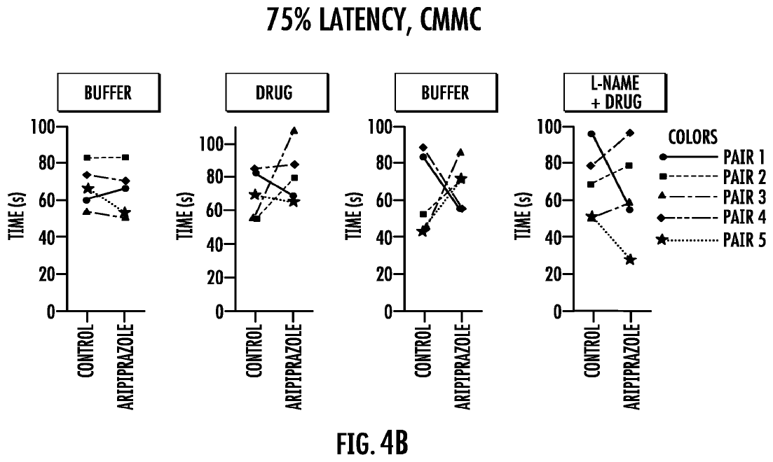

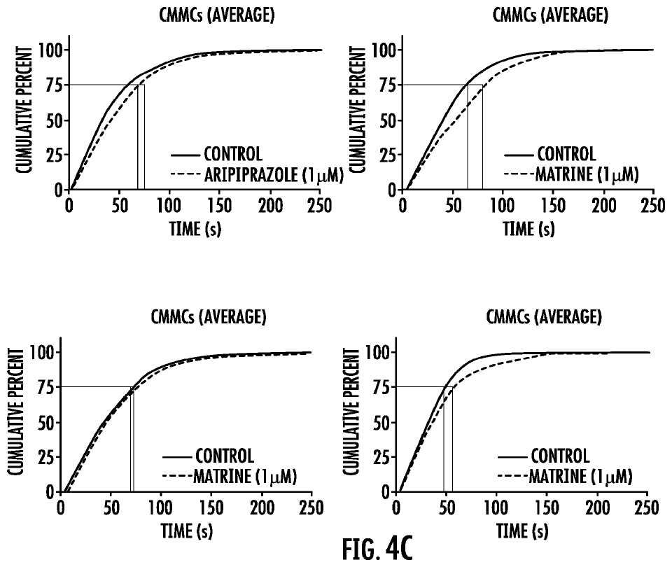

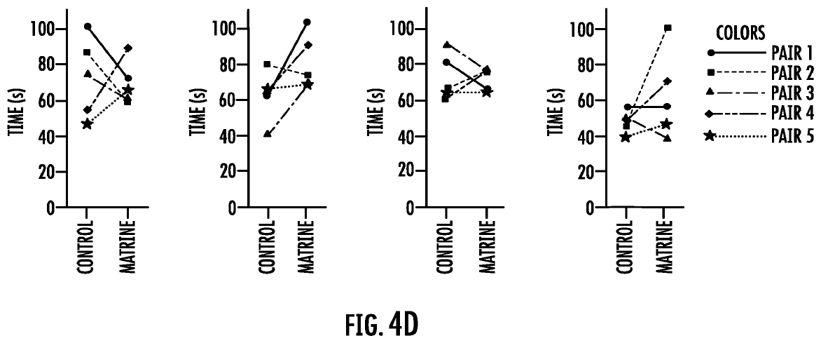

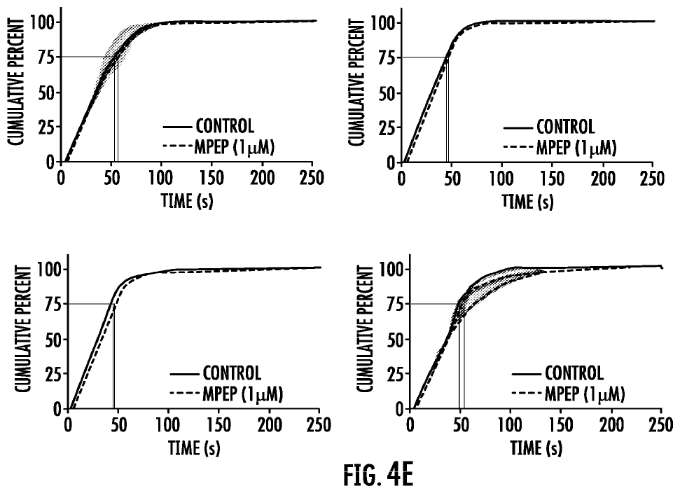

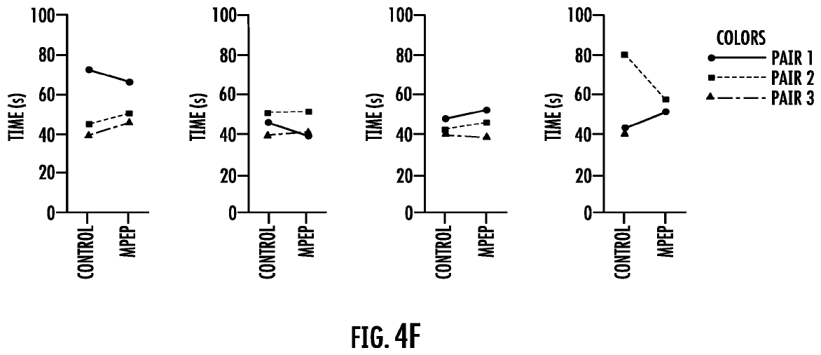

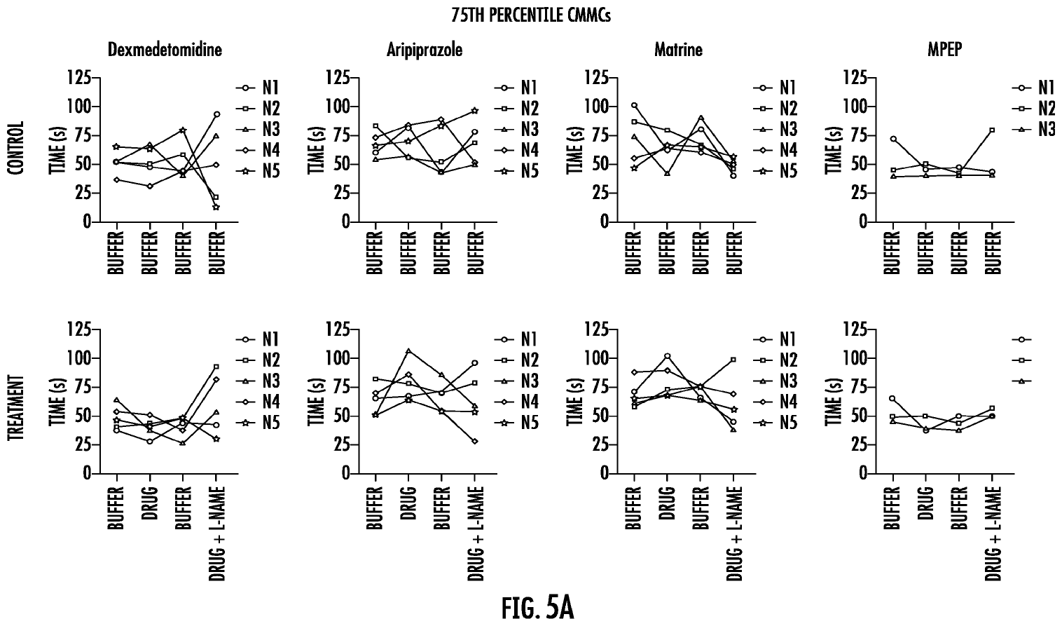

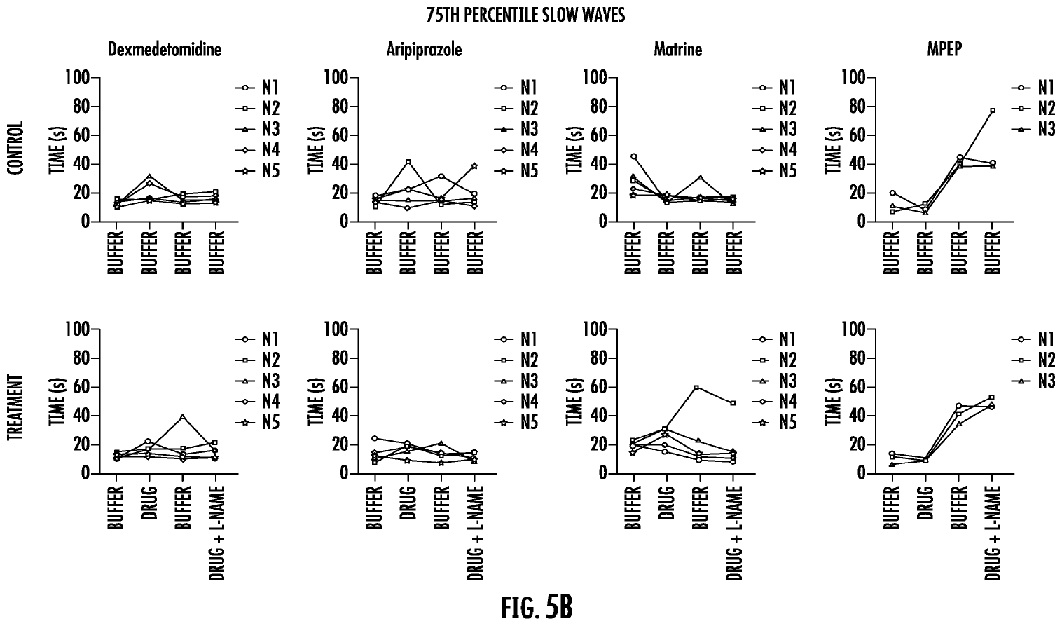

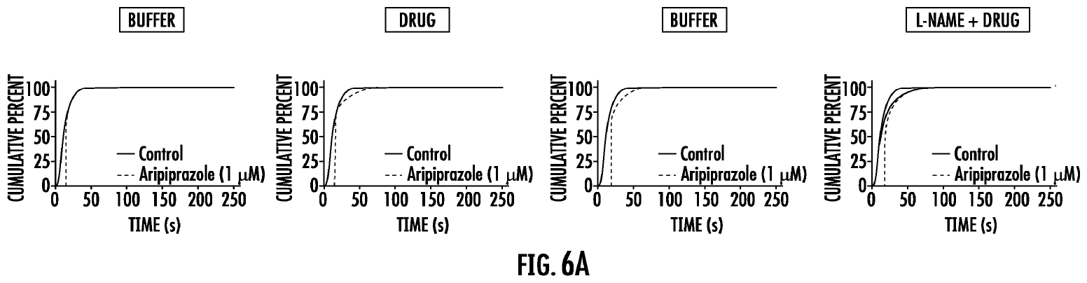

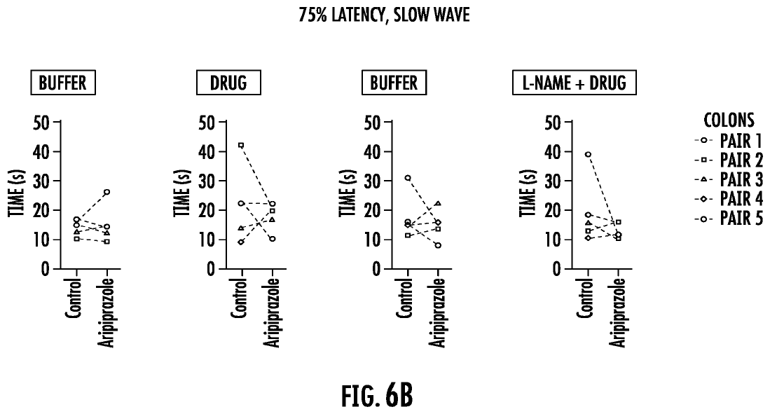

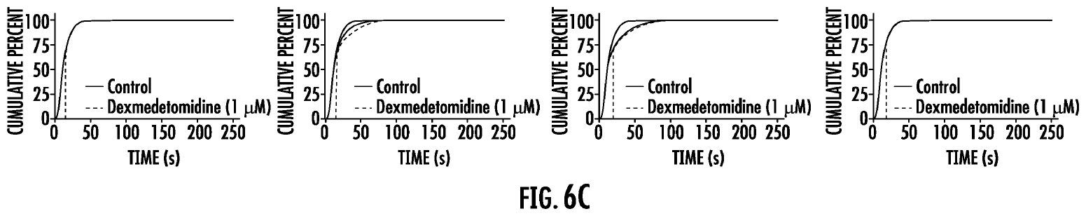

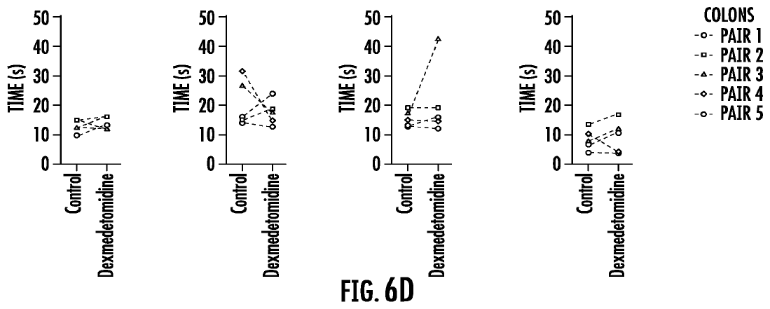

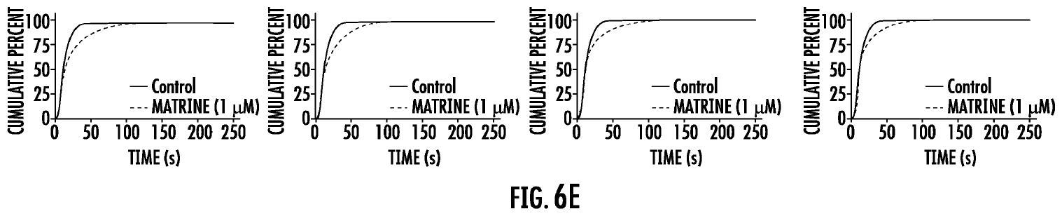

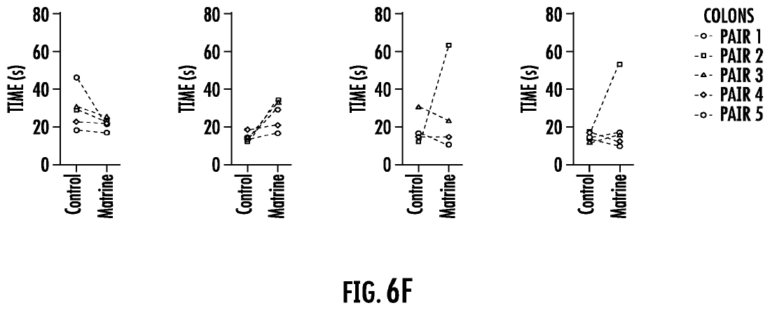

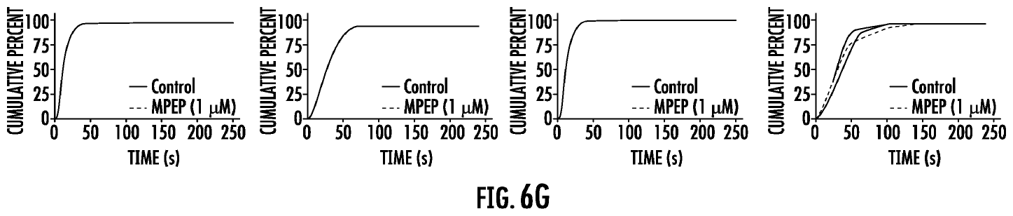

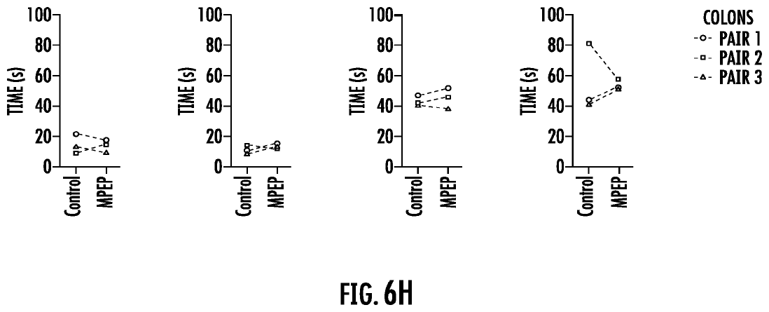

Next, a subset of hits representing different target classes was selected, prioritizing FDA approved compounds for follow up analyses (FIG. 2K and FIG. 1F). For selected compounds, a more comprehensive and integrated target analysis was performed by combining reported experimental data (Binding DB) and computational methods (SEA, Carlsbad, Dinies, Swisstarget, Superdrug, Pubchem Bioassays, FIG. 2G). The effects of these compounds was tested on colonic motility in organ bath assays (FIG. 2H). In these assays, resected segments of mouse colon were maintained in a physiologic buffer to study motility patterns using video recording. In the first experiment the effect of all selected drug candidates was tested on mouse colonic motility, ex vivo (FIG. 2I). In each experiment, an untreated control and a drug-treated colon sample were tested side by side during five consecutive 10-min acquisitions. For each acquisition, instead of analyzing fecal outputs, which are generally variable, more sophisticated contraction analysis was performed by generating spatiotemporal maps from video data based on the changes in the colonic diameter over time, and used to calculate the rate of colonic migrating motor complexes (CMMC) and slow waves (SW). CMMCs are rhythmic propulsive contractions initiated by the ENS while SWs are mediated through the pacemaking activity of the interstitial cells of Cajal (Barajas-López and Huizinga, 1989; Burns et al., 1996; Fida et al., 1997; Lyster et al., 1995; Smith et al., 1987). To probe the dynamics of CMMC and SW events during each acquisition, cumulative percent graphs were generated (FIG. 3A-B) and the intervals were calculated at the 75th percentile (FIG. 2J and FIG. 3C). Compounds that showed promising effects on lowering CMMC intervals relative to the untreated condition were chosen for follow-up assessment (i.e., aripiprazole, dexmedetomidine, matrine, MPEP) (FIG. 2J and FIG. 3A-C). To assess whether the compounds mediated their effect on CMMCs through modulating NO release, sequential drug treatments were performed in the presence and absence of the NOS1 inhibitor N (omega)-nitro-L-arginine methyl ester (L-NAME). Each experiment consisted of four 6-min acquisitions on five independent sample pairs in the control and drug-treated groups (FIG. 2K). Of the tested drugs, the adrenergic receptor agonist dexmedetomidine, decreased CMMC intervals in four out of five colon samples, an effect that was absent when colons were treated with dexmedetomidine+L-NAME simultaneously (FIG. 2L, FIG. 4A-F, and FIG. 5A-B). SWs were not affected by the drug treatment (FIG. 6A-H). In addition to CMMCs, the effects of compounds on colonic motility were quantified by annotating and measuring anterograde contractile events detected in the spatiotemporal map. These events were termed “longitudinal contractile events” (LCE) and highlighted by representative arrows in FIG. 2K. In dexmedetomidine-treated colons, a decrease in the total number of LCEs (FIG. 2M) and an increasing trend in their average duration (FIG. 2N) was observed in all five replicates. These effects were reversed after the drug removal and blocked by L-NAME co-treatment (FIG. 2M-N). These results provide a blueprint for leveraging in vitro human ENS models to uncover mechanisms that regulate GI motility which are capable of identifying therapies that target specific ENS populations.

### 3. High-Throughput Small Molecule Screen Reveals PDGFR Inhibition as a Driver of NO Neuron Induction

To evaluate the potential of hPSC-derived cultures to model human ENS development, it was determined to define the mechanism of NO neuron specification in vitro. Searching for pathways that regulate NO neuron differentiation, a high-throughput small molecule screen was performed. Identifying distinct pathways and chemical modulators that promote NO neuron induction could provide insights on NO neuron development and offer a strategy for the derivation of NO neuron enriched ENS cultures.

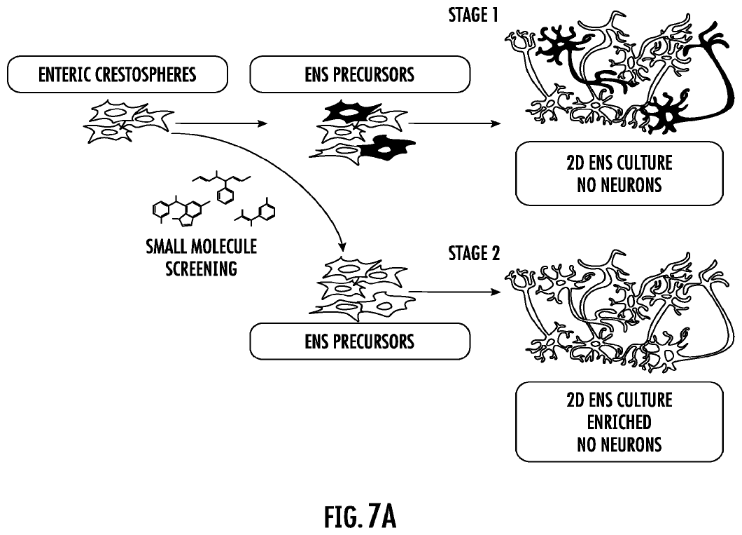

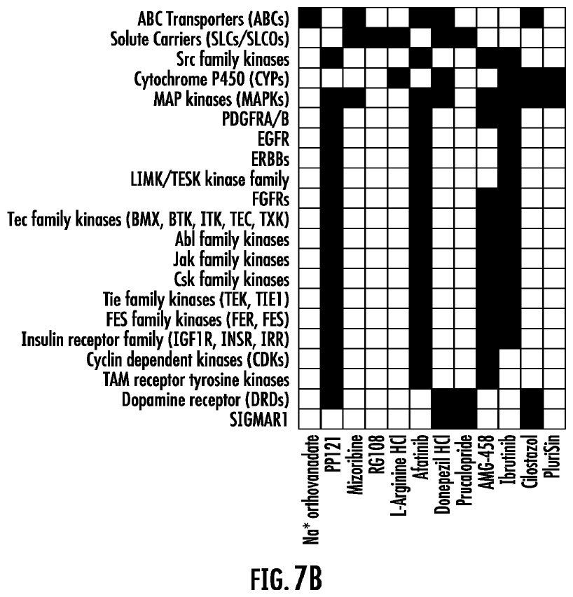

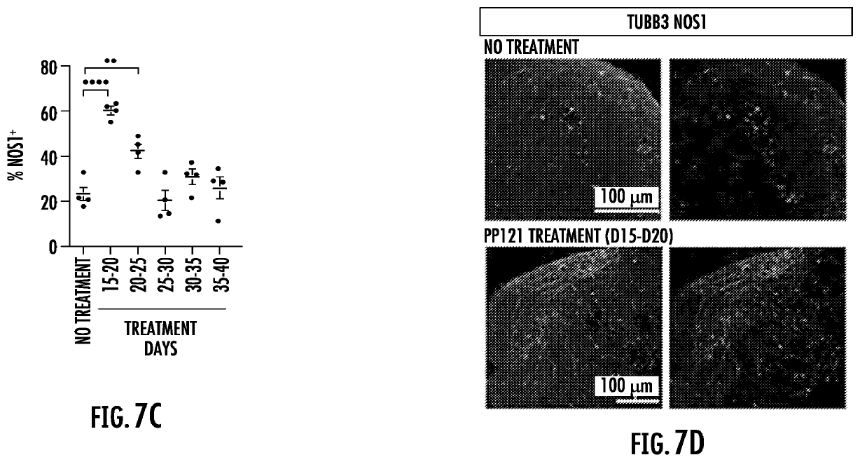

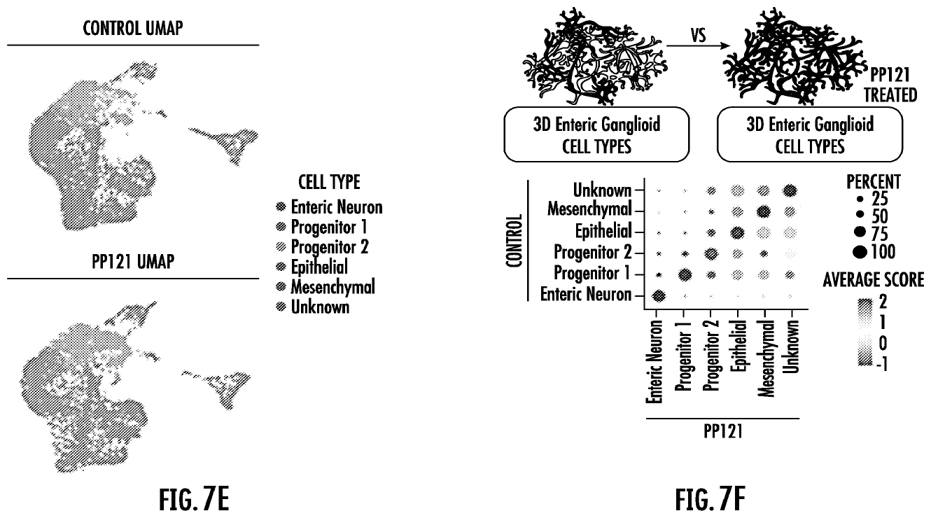

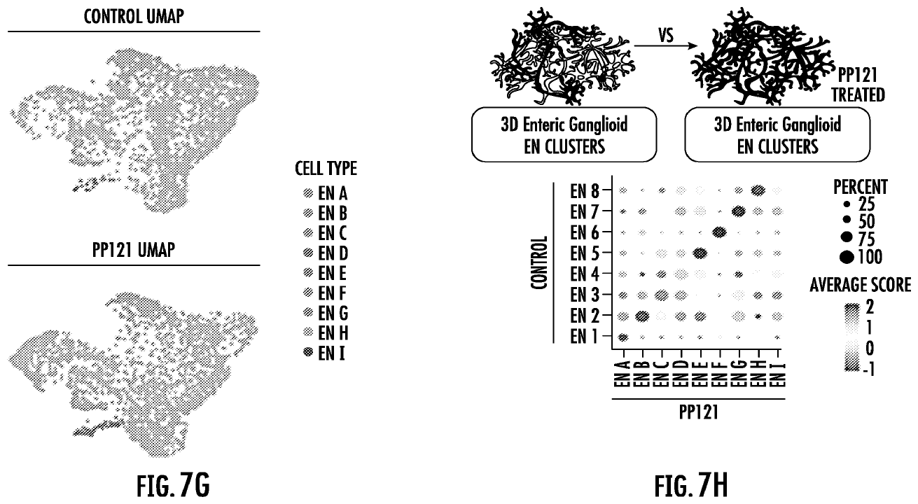

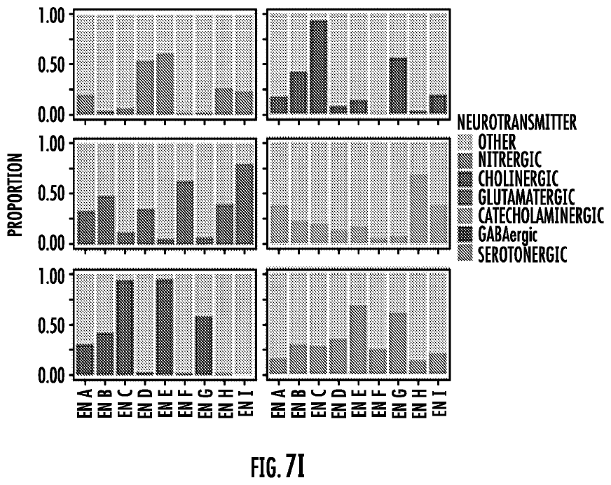

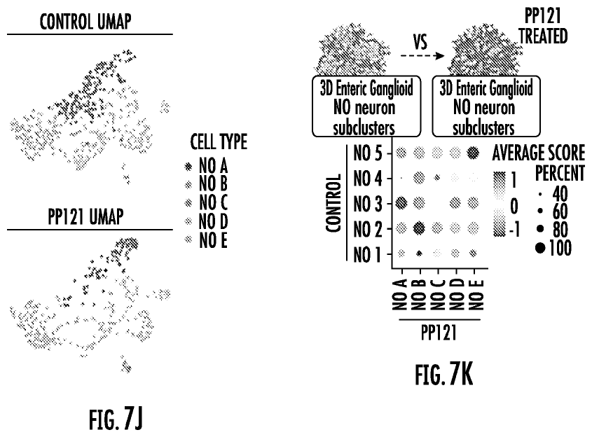

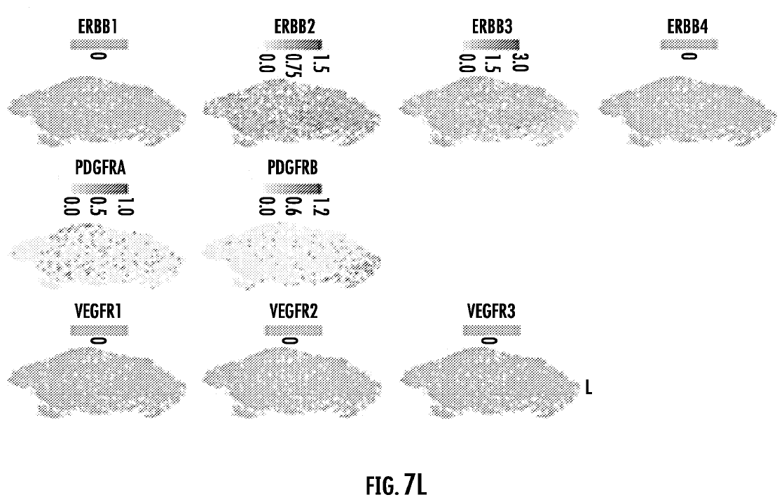

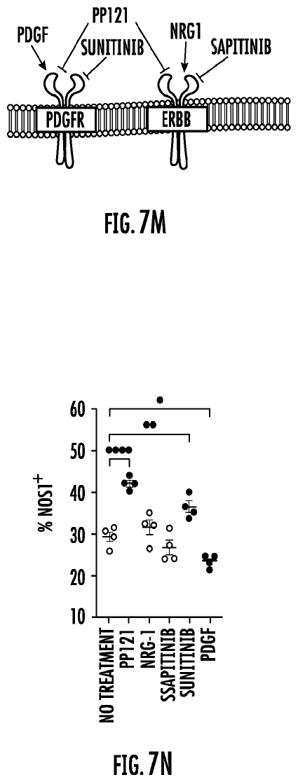

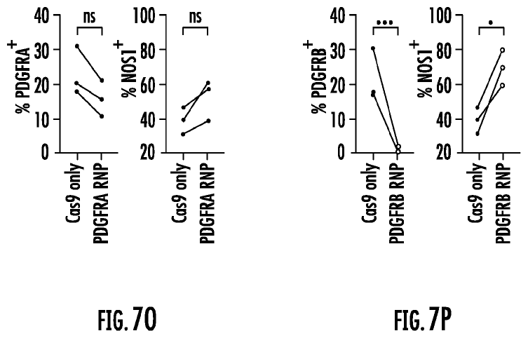

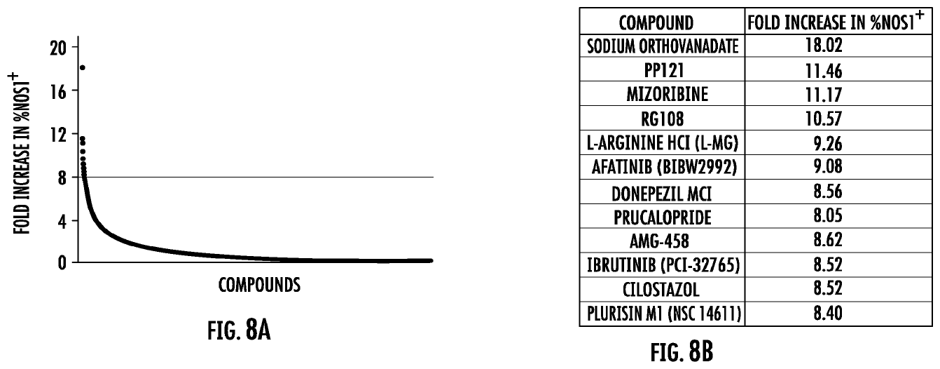

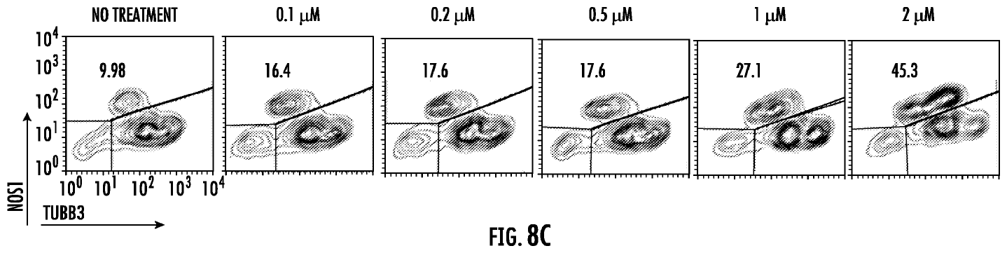

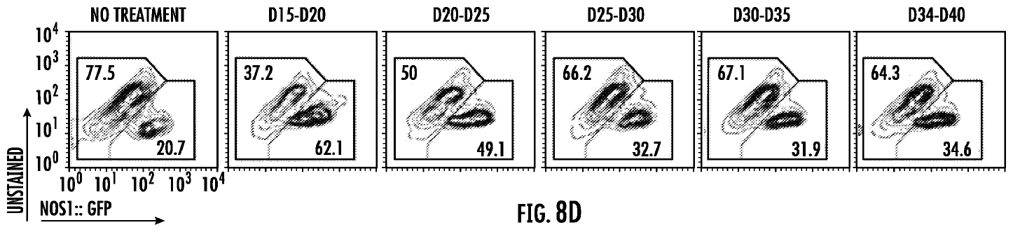

To identify compounds that induce NO neuron differentiation, enteric crestospheres were treated with 1694 compounds in the Selleck inhibitor Library™ and 12 hit compounds that increased the proportion of NOS1⁺ neurons by at least eight folds were identified (FIG. 7A and FIG. 8A-B). In order to reveal the mechanisms by which these hit compounds enhanced NO neuron induction, target prediction analysis was performed by combining reported experimental data (Binding DB) and computational methods (SEA, Carlsbad, Dinies, Swisstarget, Superdrug, Pubchem Bioassays) (FIG. 7B). After clustering the predicted protein targets, common patterns emerged for a subset of compounds. For example, PP121, ibrutinib, afatinib, and AMG-458 were all predicted to interact with EGFR, ERBBs, MAP, and TEC family kinases among others (FIG. 7B). For follow-up analysis, it was chosen to focus on PP121, the hit with the highest % NOS1⁺ fold increase in this subset of compounds. PP121 showed a dose-dependent effect on NO neuron induction efficiency as measured by flow cytometry (FIG. 8C). To find the most effective treatment window for PP121-induced NO neuron induction, the differentiating cultures were treated for five days at various time points. Measuring GFP signal in stage 1 NOS1::GFP enteric ganglioids showed the highest induction efficiency for cells treated during day 15-20 (FIG. 7C-D and FIG. 8D).

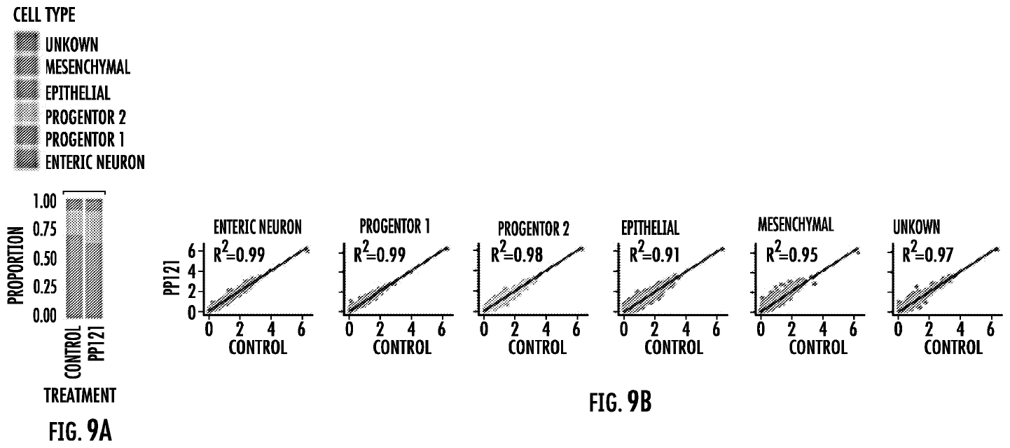

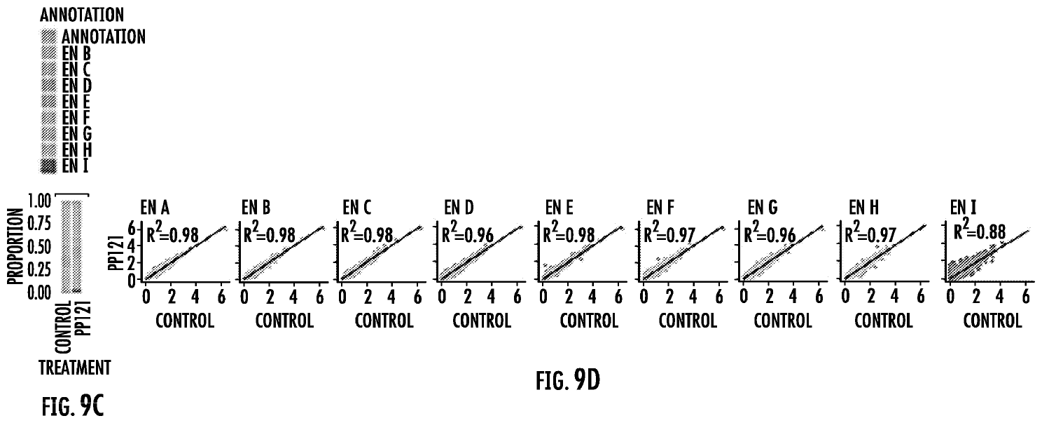

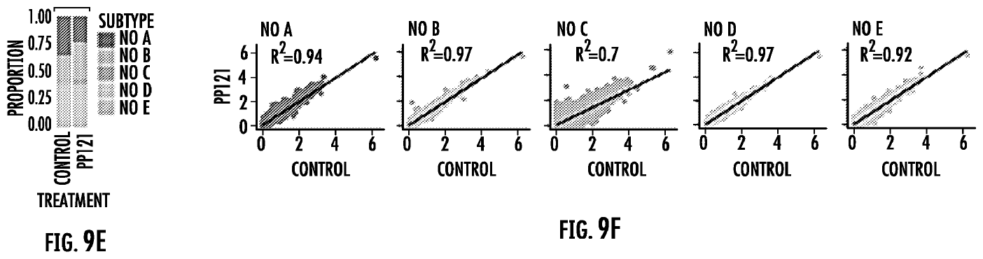

For the enrichment protocol to be reliable, it was important to confirm that PP121 treatment did not change the identity of the cell types. To compare PP121 treated and untreated stage 1 enteric ganglioids in single cell resolution, snRNA-seq was performed, and both datasets combined. This analysis revealed that all cell types were represented in both conditions (FIG. 7E-F and FIG. 9A). Importantly, comparison of the average expression of all genes for matched PP121 treated and untreated cell types showed highly similar transcriptomes (R2 correlations >0.91), indicating that PP121 treatment did not change the transcriptional identity of cell types (FIG. 9B). Interestingly, sub-clustering of the merged control and PP121 treated neurons revealed nine neuronal subtypes EN A-I (FIG. 7G). The PP121 treated dataset subtypes showed high transcriptional similarity to EN 1-8 of the control only dataset (FIG. 7H). EN cluster I consisted of mostly PP121 treated cells and few control cells and showed moderate transcriptional similarity to the control only EN cluster 4, suggesting that this neuronal subtype is present but rare in control cultures causing those neurons to cluster with the most similar subtype, EN 4 (FIG. 7H and FIG. 9C). Along with EN I which is roughly 25% nitrergic, PP121 treatment also enriched cultures for neuronal subtypes EN D and H (roughly 50% and 25% nitrergic, respectively), while EN A and G were less represented (FIG. 7I and FIG. 9C). Again, despite the changes in subtype abundance, control and PP121 treated neurons of the same subtype showed similar transcriptomes (R2 correlations >0.88) (FIG. 9D). Further sub-clustering of merged control and PP121 treated nitrergic neurons revealed an enrichment for Nitrergic B (most similar to control only Nitrergic 2) and a rare population, Nitrergic C (most similar to control only Nitrergic 3) (FIG. 7-K and FIG. 9E). Transcriptome comparison again showed highly similar gene expression of control and PP121 treated nitrergic neurons of the same subtype (R2 correlations >0.7) with the highest variance between Nitrergic C neurons, likely due to the small number of neurons in this cluster (FIG. 9F). Altogether, this data suggests that early treatment of ganglioids with PP121 causes changes in the abundance of neuronal subtypes normally found in untreated cultures without affecting the gene expression patterns of the subtypes that arise.

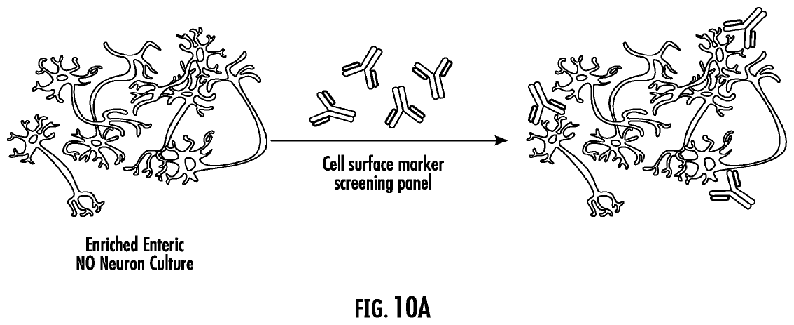

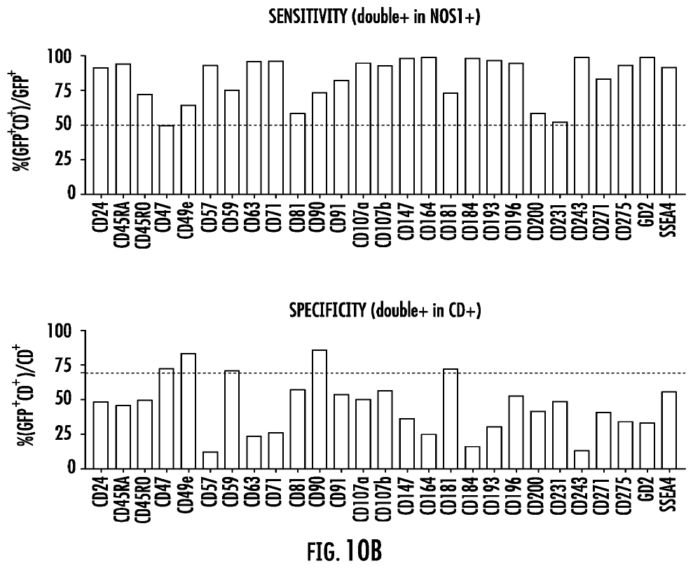

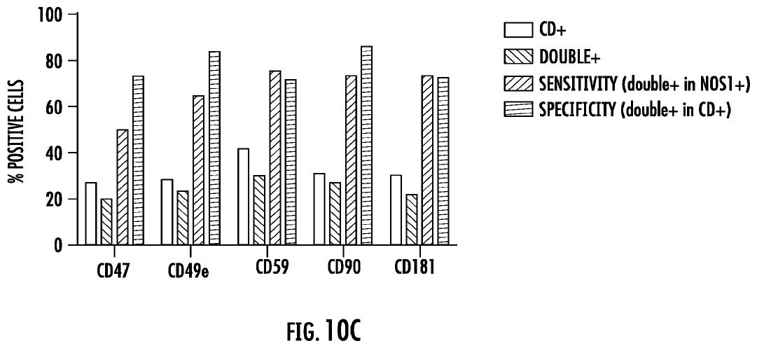

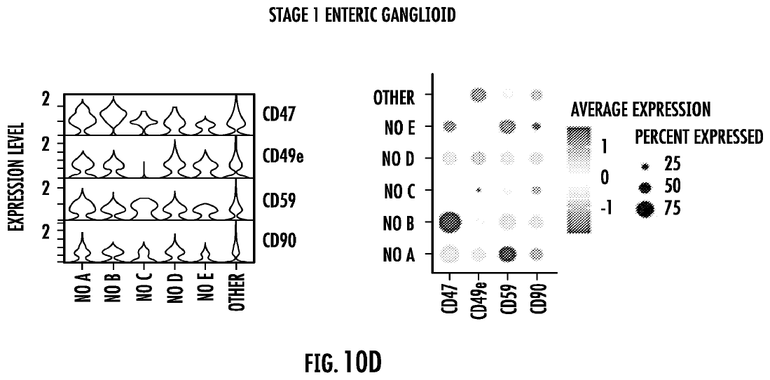

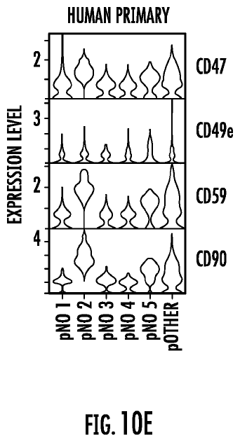

The ability to purify enteric NO neurons is of great interest especially for applications such as cell therapy. Access to NOS1::GFP reporter line and the ability to direct the differentiation towards NO neurons using PP121, allowed for FACS-compatible surface markers to be searched for these cells. A panel of 242 antibodies was screened for human cell surface molecules (BD lyoplate) and GFP and surface antigen expression signals were measured by flow cytometry (FIG. 10A). 27 antibodies were identified that stained at least 50% of NOS1 neurons (% CD⁺GFP⁺ in GFP⁺ population, FIG. 10B, top). To identify the most specific candidates among these hits, antibodies with a >70% CD⁺GFP⁺ to CD⁺ staining ratio (FIG. 10B, bottom) were identified. CD47, CD49e, CD59, CD90, and CD181 met both criteria (FIG. 10C). The expression and enrichment of CD47, CD49e, CD59, and CD90 was further confirmed in stage 1 ganglioid and NO neuron clusters in the human primary snRNA-seq dataset (FIG. 10D-E). As an example, the localization of CD47 was further confirmed in NO neurons in human primary colonic myenteric ganglia using immunohistochemistry (FIG. 10F). In addition to identifying antibodies to specifically enrich NO neurons, twelve antibodies were found that stained >70% of ganglioid cells and could serve as pan enteric neuronal surface markers (CD24, CD45RA, CD57, CD63, CD71, CD121b, CD147, CD164, CD184, CD193, CD243, CD275) (FIG. 10G). The enriched expression of CD24 was confirmed in the enteric neurons (snRNA-seq data) and also in primary human colon myenteric ganglion (FIG. 10H-I).

To determine the mechanism by which PP121 induced NO neuron enrichment in ganglioids, a combination of pharmacological and genetic approaches was used. PP121 is a multi-targeted receptor tyrosine kinase (RTK) inhibitor with known inhibitory activity on PDGFRs, VEGFRs, and EGFRs (Apsel et al., 2008). The crestosphere snRNA-seq analysis confirmed the expression of PDGFRA, PDGFRB, ERBB2, and ERBB3 while the mRNA for VEGFRs were not detectable (FIG. 7L). The induction efficiency of NO neurons was evaluated in response to PDGF (PDGFR agonist), sunitinib (PDGFR and VEGFR antagonist), NRG1 (ERBBs agonist), and sapitinib (ERBBs antagonist) (FIG. 7M). While NRG1 and sapitinib showed no significant effect on NO neuron induction, treatment with PDGF and sunitinib led to lower and higher NO neuron proportions respectively (FIG. 7N). To genetically confirm the role of PDGFR signaling in NO neuron induction, CRISPR-Cas9 was used to knock-out PDGFRA and PDGFRB in the enteric crestospheres and the percentage of NO neurons in stage 1 ganglioids was analyzed. NO neurons were enriched in both PDGFRA and PDGFRB knock-out cultures further confirming the PP121 mechanism of action (FIG. 7O-P).

4. hESC-Derived NOS1 Neurons Engraft in NOS1^{-/-} Mouse Colon

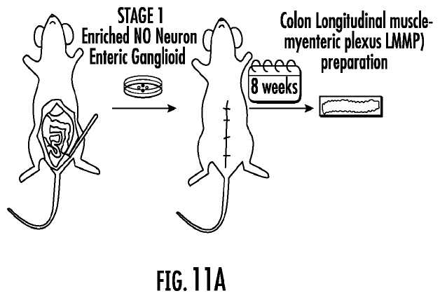

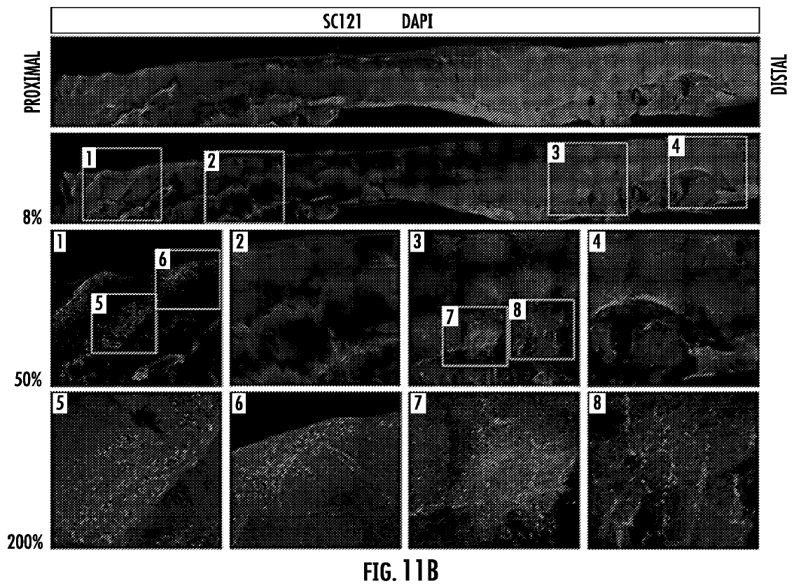

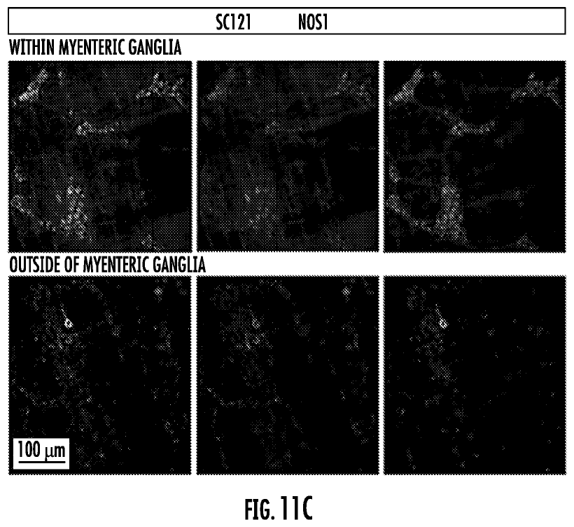

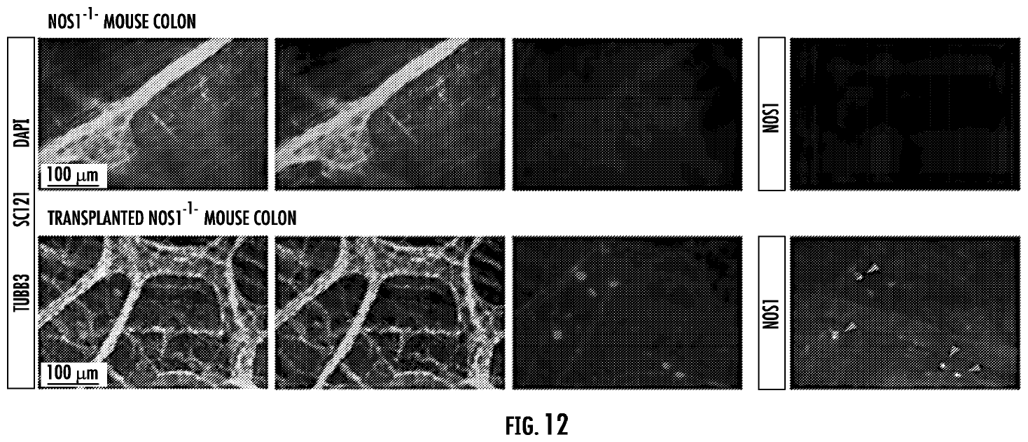

Developing an experimental system to study the human ENS in vivo opens a wide range of basic science and clinical opportunities. For example, human ENS xenografts will enable studying human neuronal circuitry in vivo and investigating ENS-CNS and ENS-immune system-microbiome communications. They also provide platforms for disease modeling and drug development. In addition, the limited regenerative capacity of the ENS highlights the importance of developing cell therapy approaches to replace the lost populations of neurons. There is currently no clinical intervention to replace the damaged or lost neurons caused by genetic and acquired ENS pathologies such as Hirschsprung disease and diabetes. It was previously shown that hPSC-derived ENC precursors can successfully engraft in vivo (Fattahi et al., 2016). McCann et al. have also shown the transplanted ex vivo cultured murine enteric neurospheres are able to rescue GI motility defects in Nos1^{-/-} mice (McCann et al., 2017). However, these neurospheres are heterogeneous populations containing only a small percentage of NO neurons. Additionally, obtaining sufficient numbers of neurospheres from human primary tissue poses a significant limitation for ultimate regenerative applications. Compared to ENC precursors, transplanting mature neurons provides a post-mitotic source of cells with a lower clinical risk of tumor formation. Obtaining highly enriched NO neuron cultures encouraged the transplantation potential of the enteric ganglioids to be assessed. PP121 treated enteric ganglioids were injected in the wall of distal colon in immunocompromised Nos1^{-/-} (B6.129S4-Nos1^{tm1Plh/J}) mice. Animals were sacrificed eight weeks post-surgery and colonic longitudinal muscle myenteric plexus (LMMP) preparations were assessed by fluorescence microscopy (FIG. 11A). Transplanted cells were distinguished by the expression of human cytoplasmic marker SC121. Notably, a remarkable number of SC121⁺ cells that had integrated along the length of the colon were observed (FIG. 11B). Engrafted cells were detected within, and outside of myenteric ganglia and many expressed NOS1 confirming their NO fate (FIG. 11C and FIG. 12). In addition to the clinically-significant cell therapy application, the developed human enteric ganglioid xenograft offers previously unachievable opportunities towards understanding development, physiology and pathophysiology of the human ENS in vivo.

### 5. Discussion

An exceptional advantage of hPSC-derived cultures is their scalability. This is particularly important when the desired cell types are rare, and have very limited regenerative and proliferative capacity such as nervous tissue. The ENS culture platforms used herein have repeatedly proven to be reliable in providing scalable sources of ENS cell types that are compatible with applications that would otherwise be extremely challenging to implement, such as high-throughput screens. In particular, using a 2D ENS cultures thousands of inhibitors were screened to identify compounds that direct the differentiation towards the clinically valuable NO neurons. Investigating the mechanism of action of the top hits revealed pathways that are important in NO neuron fate specification. Using a combination of pharmacological and genetic approaches, the contribution of one such pathway, PDGFR signaling, in inducing NO neurons was discovered, which highlights the remarkable potential of hPSC-based platforms to uncover developmental mechanisms.

Herein, functional screening platforms have been used to uncover candidate drugs that specifically modulate the activity of NO neurons. Interestingly, the hit compounds commonly target adrenergic, cholinergic, and serotonergic receptors, and sodium channels. Notably, these targets are overrepresented in NO neurons, highlighting the specificity of these compounds and their potential for further therapeutic development for GI indications. By testing a subset of these neuromodulators, it was further demonstrated that these candidate drugs are capable of affecting colonic motility patterns in ex vivo organ bath assays. This is the first example of identifying candidate drugs for modulating GI motility by targeting a specific enteric neuron subtype. These findings showcase the reliability, robustness, and scalability of hPSC derived ENS models.

### G. References

Alhawaj, A. F. (2021). Stem cell-based therapy for hirschsprung disease, do we have the guts to treat? Gene Ther.
Apsel, B., Blair, J. A., Gonzalez, B., Nazif, T. M., Feldman, M. E., Aizenstein, B., Hoffman, R., Williams, R. L., Shokat, K. M., and Knight, Z. A. (2008). Targeted polypharmacology: discovery of dual inhibitors of tyrosine and phosphoinositide kinases. Nat. Chem. Biol. 4, 691-699.
Baetge, G., and Gershon, M. D. (1989). Transient catecholaminergic (TC) cells in the vagus nerves and bowel of fetal mice: relationship to the development of enteric neurons. Dev. Biol. 132, 189-211.
Baetge, G., Pintar, J. E., and Gershon, M. D. (1990). Transiently catecholaminergic (TC) cells in the bowel of the fetal rat: precursors of noncatecholaminergic enteric neurons. Dev. Biol. 141, 353-380.
Barajas-López, C., and Huizinga, J. D. (1989). Different mechanisms of contraction generation in circular muscle of canine colon. Am. J. Physiol. 256, G570-580.
Barber, K., Studer, L., and Fattahi, F. (2019). Derivation of enteric neuron lineages from human pluripotent stem cells. Nat. Protoc. 14, 1261-1279.
Bergner, A. J., Stamp, L. A., Gonsalvez, D. G., Allison, M. B., Olson, D. P., Myers, M. G., Anderson, C. R., and Young, H. M. (2014). Birthdating of myenteric neuron subtypes in the small intestine of the mouse. J. Comp. Neurol. 522, 514-527.
Bódi, N., Szalai, Z., and Bagyánszki, M. (2019). Nitrergic Enteric Neurons in Health and Disease-Focus on Animal Models. Int. J. Mol. Sci. 20, E2003.
Bondurand, N., and Southard-Smith, E. M. (2016). Mouse models of Hirschsprung disease and other developmental disorders of the enteric nervous system: Old and new players. Dev. Biol. 417, 139-157.
Bredt, D. S., Hwang, P. M., and Snyder, S. H. (1990). Localization of nitric oxide synthase indicating a neural role for nitric oxide. Nature 347, 768-770.
Brehmer, A. (2021). Classification of human enteric neurons. Histochem. Cell Biol. 156, 95-108.
Bullitt, E. (1990). Expression of c-fos-like protein as a marker for neuronal activity following noxious stimulation in the rat. J. Comp. Neurol. 296, 517-530.
Bult, H., Boeckxstaens, G. E., Pelckmans, P. A., Jordaens, F. H., Van Maercke, Y. M., and Herman, A. G. (1990). Nitric oxide as an inhibitory non-adrenergic non-cholinergic neurotransmitter. Nature 345, 346-347.
Burns, A. J., and Douarin, N. M. (1998). The sacral neural crest contributes neurons and glia to the post-umbilical gut: spatiotemporal analysis of the development of the enteric nervous system. Dev. Camb. Engl. 125, 4335-4347.
Burns, A. J., Lomax, A. E., Torihashi, S., Sanders, K. M., and Ward, S. M. (1996). Interstitial cells of Cajal mediate inhibitory neurotransmission in the stomach. Proc. Natl. Acad. Sci. U.S.A 93, 12008-12013.
Burns, A. J., Goldstein, A. M., Newgreen, D. F., Stamp, L., Schäfer, K.-H., Metzger, M., Hotta, R., Young, H. M., Andrews, P. W., Thapar, N., et al. (2016). White paper on guidelines concerning enteric nervous system stem cell therapy for enteric neuropathies. Dev. Biol. 417, 229-251.
Camilleri, M. (2021). Gastrointestinal motility disorders in neurologic disease. J. Clin. Invest. 131, 143771.
Camilleri, M., Bharucha, A. E., and Farrugia, G. (2011). Epidemiology, mechanisms, and management of diabetic gastroparesis. Clin. Gastroenterol. Hepatol. Off. Clin. Pract. J. Am. Gastroenterol. Assoc. 9, 5-12; quiz e7.
De Giorgio, R. (2006). The Enteric Nervous System, J. B. Furness. Blackwell Publishing, Oxford (2006), (274 pp., Price GBP 55.00, ISBN 1-4051-3376-7). Dig. Liver Dis. 38, 441.
De Giorgio, R., Bianco, F., Latorre, R., Caio, G., Clavenzani, P., and Bonora, E. (2016). Enteric neuropathies: Yesterday, Today and Tomorrow. Adv. Exp. Med. Biol. 891, 123-133.
Drokhlyansky, E., Smillie, C. S., Van Wittenberghe, N., Ericsson, M., Griffin, G. K., Eraslan, G., Dionne, D., Cuoco, M. S., Goder-Reiser, M. N., Sharova, T., et al. (2020). The Human and Mouse Enteric Nervous System at Single-Cell Resolution. Cell 182, 1606-1622.e23.
Fattahi, F., Steinbeck, J. A., Kriks, S., Tchieu, J., Zimmer, B., Kishinevsky, S., Zeltner, N., Mica, Y., El-Nachef, W., Zhao, H., et al. (2016). Deriving human ENS lineages for cell therapy and drug discovery in Hirschsprung disease. Nature 531, 105-109.
Fida, R., Lyster, D. J., Bywater, R. A., and Taylor, G. S. (1997). Colonic migrating motor complexes (CMMCs) in the isolated mouse colon. Neurogastroenterol. Motil. Off. J. Eur. Gastrointest. Motil. Soc. 9, 99-107.
Fung, C., and Vanden Berghe, P. (2020). Functional circuits and signal processing in the enteric nervous system. Cell. Mol. Life Sci. CMLS 77, 4505-4522.
Furness, J. B., Callaghan, B. P., Rivera, L. R., and Cho, H.-J. (2014). The enteric nervous system and gastrointestinal innervation: integrated local and central control. Adv. Exp. Med. Biol. 817, 39-71.
Gershon, M. D. (1999). The enteric nervous system: a second brain. Hosp. Pract. 1995 34, 31-32, 35-38, 41-42 passim.
Grubišić, V., and Gulbransen, B. D. (2017). Enteric glia: the most alimentary of all glia. J. Physiol. 595, 557-570.
Grundmann, D., Loris, E., Maas-Omlor, S., Huang, W., Scheller, A., Kirchhoff, F., and Schäfer, K.-H. (2019). Enteric Glia: S100, GFAP, and Beyond. Anat. Rec. Hoboken NJ 2007 302, 1333-1344.
Hamnett, R., Dershowitz, L. B., Sampathkumar, V., Wang, Z., Andrade, V. D., Kasthuri, N., Druckmann, S., and Kaltschmidt, J. A. (2021). Regional cytoarchitecture of the adult and developing mouse enteric nervous system.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29.
Heanue, T. A., and Pachnis, V. (2007). Enteric nervous system development and Hirschsprung's disease: advances in genetic and stem cell studies. Nat. Rev. Neurosci. 8, 466-479.
Hunt, S. P., Pini, A., and Evan, G. (1987). Induction of c-fos-like protein in spinal cord neurons following sensory stimulation. Nature 328, 632-634.
Lai, F. P.-L., Lau, S.-T., Wong, J. K.-L., Gui, H., Wang, R. X., Zhou, T., Lai, W. H., Tse, H.-F., Tam, P. K.-H., Garcia-Barcelo, M.-M., et al. (2017). Correction of Hirschsprung-Associated Mutations in Human Induced Pluripotent Stem Cells Via Clustered Regularly Interspaced Short Palindromic Repeats/Cas9, Restores Neural Crest Cell Function. Gastroenterology 153, 139153.e8.
Lake, J. I., and Heuckeroth, R. O. (2013). Enteric nervous system development: migration, differentiation, and disease. Am. J. Physiol.-Gastrointest. Liver Physiol. 305, G1-G24.
Long-Smith, C., O'Riordan, K. J., Clarke, G., Stanton, C., Dinan, T. G., and Cryan, J. F. (2020). Microbiota-Gut-Brain Axis: New Therapeutic Opportunities. Annu. Rev. Pharmacol. Toxicol. 60, 477-502.
Lyster, D. J., Bywater, R. A., and Taylor, G. S. (1995). Neurogenic control of myoelectric complexes in the mouse isolated colon. Gastroenterology 108, 1371-1378.
McCann, C. J., Cooper, J. E., Natarajan, D., Jevans, B., Burnett, L. E., Burns, A. J., and Thapar, N. (2017). Transplantation of enteric nervous system stem cells rescues nitric oxide synthase deficient mouse colon. Nat. Commun. 8, 15937.
Morarach, K., Mikhailova, A., Knoflach, V., Memic, F., Kumar, R., Li, W., Ernfors, P., and Marklund, U. (2021). Diversification of molecularly defined myenteric neuron classes revealed by single-cell RNA sequencing. Nat. Neurosci. 24, 34-46.
Muller, P. A., Koscsó, B., Rajani, G. M., Stevanovic, K., Berres, M.-L., Hashimoto, D., Mortha, A., Leboeuf, M., Li, X.-M., Mucida, D., et al. (2014). Crosstalk between muscularis macrophages and enteric neurons regulates gastrointestinal motility. Cell 158, 300-313.
Nagy, N., and Goldstein, A. M. (2017). Enteric nervous system development: A crest cell's journey from neural tube to colon. Semin. Cell Dev. Biol. 66, 94-106.
Niesler, B., Kuerten, S., Demir, I. E., and Schäfer, K.-H. (2021). Disorders of the enteric nervous system-a holistic view. Nat. Rev. Gastroenterol. Hepatol. 18, 393-410.
Obata, Y., and Pachnis, V. (2016). The Effect of Microbiota and the Immune System on the Development and Organization of the Enteric Nervous System. Gastroenterology 151, 836-844.
Obermayr, F., Stamp, L. A., Anderson, C. R., and Young, H. M. (2013). Genetic fate-mapping of tyrosine hydroxylase-expressing cells in the enteric nervous system. Neurogastroenterol. Motil. Off. J. Eur. Gastrointest. Motil. Soc. 25, e283-291.
Paridaen, J. T. M. L., and Huttner, W. B. (2014). Neurogenesis during development of the vertebrate central nervous system. EMBO Rep. 15, 351-364.
Pesce, M., Borrelli, O., Saliakellis, E., and Thapar, N. (2018). Gastrointestinal Neuropathies: New Insights and Emerging Therapies. Gastroenterol. Clin. North Am. 47, 877-894.
Qu, Z.-D., Thacker, M., Castelucci, P., Bagyánszki, M., Epstein, M. L., and Furness, J. B. (2008). Immunohistochemical analysis of neuron types in the mouse small intestine. Cell Tissue Res. 334, 147-161.
Rao, M., and Gershon, M. D. (2018). Enteric nervous system development: what could possibly go wrong? Nat. Rev. Neurosci. 19, 552-565.
Rivera, L. R., Poole, D. P., Thacker, M., and Furness, J. B. (2011). The involvement of nitric oxide synthase neurons in enteric neuropathies. Neurogastroenterol. Motil. Off. J. Eur. Gastrointest. Motil. Soc. 23, 980-988.
Rothman, T. P., Tennyson, V. M., and Gershon, M. D. (1986). Colonization of the bowel by the precursors of enteric glia: studies of normal and congenitally aganglionic mutant mice. J. Comp. Neurol. 252, 493-506.
Rowitch, D. H., and Kriegstein, A. R. (2010). Developmental genetics of vertebrate glial-cell specification. Nature 468, 214-222.
Santos, P. L., Brito, R. G., Matos, J. P. S. C. F., Quintans, J. S. S., and Quintans-Júnior, L. J. (2018). Fos Protein as a Marker of Neuronal Activity: a Useful Tool in the Study of the Mechanism of Action of Natural Products with Analgesic Activity. Mol. Neurobiol. 55, 4560-4579.
Sasselli, V., Pachnis, V., and Burns, A. J. (2012). The enteric nervous system. Dev. Biol. 366, 64-73.
Schneider, S., Wright, C. M., and Heuckeroth, R. O. (2019). Unexpected Roles for the Second Brain: Enteric Nervous System as Master Regulator of Bowel Function. Annu. Rev. Physiol. 81, 235-259.
Serbedzija, G. N., Burgan, S., Fraser, S. E., and Bronner-Fraser, M. (1991). Vital dye labelling demonstrates a sacral neural crest contribution to the enteric nervous system of chick and mouse embryos. Dev. Camb. Engl. 111, 857-866.
Smith, T. K., Reed, J. B., and Sanders, K. M. (1987). Origin and propagation of electrical slow waves in circular muscle of canine proximal colon. Am. J. Physiol. 252, C215-224.
Spear, E. T., Holt, E. A., Joyce, E. J., Haag, M. M., Mawe, S. M., Hennig, G. W., Lavoie, B., Applebee, A. M., Teuscher, C., and Mawe, G. M. (2018). Altered gastrointestinal motility involving autoantibodies in the experimental autoimmune encephalomyelitis model of multiple sclerosis. Neurogastroenterol. Motil. Off. J. Eur. Gastrointest. Motil. Soc. 30, e13349.
Steinbeck, J. A., Jaiswal, M. K., Calder, E. L., Kishinevsky, S., Weishaupt, A., Toyka, K. V., Goldstein, P. A., and Studer, L. (2016). Functional Connectivity under Optogenetic Control Allows Modeling of Human Neuromuscular Disease. Cell Stem Cell 18, 134-143.
Swaminathan, M., Hill-Yardin, E., Ellis, M., Zygorodimos, M., Johnston, L. A., Gwynne, R. M., and Bornstein, J. C. (2016). Video Imaging and Spatiotemporal Maps to Analyze Gastrointestinal Motility in Mice. J. Vis. Exp. JoVE 53828.
Ward, S. M., Xue, C., Shuttleworth, C. W., Bredt, D. S., Snyder, S. H., and Sanders, K. M. (1992). NADPH diaphorase and nitric oxide synthase colocalization in enteric neurons of canine proximal colon. Am. J. Physiol. 263, G277-284.
Wu, Y., Tamayo, P., and Zhang, K. (2018). Visualizing and Interpreting Single-Cell Gene Expression Datasets with Similarity Weighted Nonnegative Embedding. Cell Syst. 7, 656666.e4.
Yarandi, S. S., and Srinivasan, S. (2014). Diabetic gastrointestinal motility disorders and the role of enteric nervous system: current status and future directions. Neurogastroenterol. Motil. Off. J. Eur. Gastrointest. Motil. Soc. 26, 611-624.
Yoo, B. B., and Mazmanian, S. K. (2017). The Enteric Network: Interactions between the Immune and Nervous Systems of the Gut. Immunity 46, 910-926.
Young, H. M., Furness, J. B., Shuttleworth, C. W., Bredt, D. S., and Snyder, S. H. (1992). Co-localization of nitric oxide synthase immunoreactivity and NADPH diaphorase staining in neurons of the guinea-pig intestine. Histochemistry 97, 375-378.
Young, H. M., Bergner, A. J., and Müller, T. (2003). Acquisition of neuronal and glial markers by neural crest-derived cells in the mouse intestine. J. Comp. Neurol. 456, 1-11.

It will be apparent to those skilled in the art that various modifications and variations can be made in the present invention without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1.-19. (canceled)

20. A method for treating a gut motility disorder in a subject in need thereof, the method comprising administering to the subject an effective amount of at least one modulator of NO neuron activity.

21. The method of claim 20, wherein the at least one modulator of NO neuron activity is selected from modulators of COX, modulators of dopamine receptors, modulators of sodium channels, modulators of serotonin receptors, modulators of acetylcholine receptors, modulators of GABA receptors, modulators of FAAH, modulators of adrenergic receptors, modulators of histamine receptors, modulators of vasopressin receptors, modulators of NMDAR, modulators of beta amyloid, modulators of gamma-secretase, modulators of IxB/IKK, modulators of glutamate receptors, modulators of opioid receptors, modulators of TRPV, modulators of aldose reductase, modulators of calcium channels, modulators of glucocorticoid receptors, modulators of HMG-COA reductase, and pharmaceutically acceptable salts of the foregoing.

22. The method of claim 20, wherein the at least one modulator of NO neuron activity is selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HC1, ADX-47273, MPEP, nefopam HCl, phenazopyridine HCl, epinephrine, (+)-matrine, phenothiazine, naproxen sodium, AMG-517, isoliquiritigenin, nilvadipine, prednisone, simvastatin, and pharmaceutically acceptable salts of the foregoing.

23. The method of claim 20, wherein the at least one modulator of NO neuron activity is selected from aripiprazole, dexmedetomidine, matrine, MPEP, and pharmaceutically acceptable salts of the foregoing.

24. The method of claim 20, wherein the at least one modulator of NO neuron activity is dexmedetomidine.

25. The method of claim 20, wherein the gut motility disorder is selected from achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

26. The method of claim 20, wherein the subject is a mammal.

27. The method of claim 20, wherein the subject is a human.

28. The method of claim 20, wherein the subject has been diagnosed with a need for treatment of the gut motility disorder prior to the administering step.

29. The method of claim 20, further comprising identifying the subject in need of treatment of a gut motility disorder.

30. The method of claim 20, wherein the effective amount is a therapeutically effective amount.

31. The method of claim 20, wherein the effective amount is a prophylactically effective amount.

32. A method for modulating NO neuron activity in a subject having a gut motility disorder, the method comprising administering to the subject an effective amount of at least one modulator of NO neuron activity.

33. The method of claim 32, wherein the at least one modulator of NO neuron activity is selected from modulators of COX, modulators of dopamine receptors, modulators of sodium channels, modulators of serotonin receptors, modulators of acetylcholine receptors, modulators of GABA receptors, modulators of FAAH, modulators of adrenergic receptors, modulators of histamine receptors, modulators of vasopressin receptors, modulators of NMDAR, modulators of beta amyloid, modulators of gamma-secretase, modulators of IxB/IKK, modulators of glutamate receptors, modulators of opioid receptors, modulators of TRPV, modulators of aldose reductase, modulators of calcium channels, modulators of glucocorticoid receptors, modulators of HMG-COA reductase, and pharmaceutically acceptable salts of the foregoing.

34. The method of claim 32, wherein the at least one modulator of NO neuron activity is selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HC1, ADX-47273, MPEP, nefopam HCl, phenazopyridine HCl, epinephrine, (+)-matrine, phenothiazine, naproxen sodium, AMG-517, isoliquiritigenin, nilvadipine, prednisone, simvastatin, and pharmaceutically acceptable salts of the foregoing.

35. The method of claim 32, wherein the at least one modulator of NO neuron activity is selected from aripiprazole, dexmedetomidine, matrine, and MPEP, or a pharmaceutically acceptable salt thereof.

36. The method of claim 32, wherein the at least one modulator of NO neuron activity is dexmedetomidine.

37. The method of claim 32, wherein the modulating NO neuron activity induces colonic activity.

38. A kit comprising at least one modulator of NO neuron activity and one or a combination chosen from (a) an agent for treating a gut motility disorder; (b) instructions for treating the gut motility disorder; and (c) instructions for administering the at least one modulator of NO neuron activity in connection with treating the gut motility disorder.

39. The kit of claim 38, wherein the at least one modulator of NO neuron activity is selected from modulators of COX, modulators of dopamine receptors, modulators of sodium channels, modulators of serotonin receptors, modulators of acetylcholine receptors, modulators of GABA receptors, modulators of FAAH, modulators of adrenergic receptors, modulators of histamine receptors, modulators of vasopressin receptors, modulators of NMDAR, modulators of beta amyloid, modulators of gamma-secretase, modulators of IxB/IKK, modulators of glutamate receptors, modulators of opioid receptors, modulators of TRPV, modulators of aldose reductase, modulators of calcium channels, modulators of glucocorticoid receptors, modulators of HMG-CoA reductase, and pharmaceutically acceptable salts of the foregoing.

40. The kit of claim 38, wherein the at least one modulator of NO neuron activity is selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HC1, ADX-47273, MPEP, nefopam HCl, phenazopyridine HCl, epinephrine, (+)-matrine, phenothiazine, naproxen sodium, AMG-517, isoliquiritigenin, nilvadipine, prednisone, simvastatin, and pharmaceutically acceptable salts of the foregoing.

41. The kit of claim 38, wherein the agent is selected from a parasympathomimetic, a prokinetic agent, an opiod antagonist, an antidarrheal, and an antibiotic.

42. The kit of claim 38, wherein the agent is selected from neostigmine, bethanechol, metoclopramide, cisapride, and loperamide.

43. The kit of claim 38, wherein the at least one modulator of NO neuron activity and the agent are co-packaged.

44. The kit of claim 38, wherein the at least one modulator of NO neuron activity and the agent are co-formulated.
